(19) United States
(12) Patent Application Publication
(10) Pub. No.: US 2013/0196320 A1
(43) Pub. Date: Aug. 1, 2013

(54) METHOD FOR IMPROVING CLEAVAGE OF DNA BY ENDONUCLEASE SENSITIVE TO METHYLATION

(71) Applicants: Philippe Duchateau, Draveil (FR); Julien Valton, Paris (FR); Fayza Daboussi, Chelles (FR)

(72) Inventors: Philippe Duchateau, Draveil (FR); Julien Valton, Paris (FR); Fayza Daboussi, Chelles (FR)

(73) Assignee: CELLECTIS, Paris (FR)

(21) Appl. No.: 13/704,417

(22) PCT Filed: Jun. 15, 2011
(86) PCT No.: PCT/IB2011/002196

§ 371 Date: Mar. 22, 2013

Related U.S. Application Data
(60) Provisional application No. 61/354,923, filed on Jun. 15, 2010.
(60) Provisional application No. 61/382,773, filed on Sep. 14, 2010.
(60) Provisional application No. 61/484,005, filed on May 9, 2011.

(51) Int. Cl. C12P 19/34

(52) U.S. Cl. CPC C12P 19/34
USPC 435/6.11; 536/23.1; 435/320.1; 435/252.33; 435/196

(57) ABSTRACT

The present invention concerns novel methods for improving cleavage of DNA by rare-cutting endonucleases, overcoming DNA modification constraints, particularly DNA methylation, thereby giving new tools for genome engineering, particularly to increase the integration efficiency of a transgene into a genome at a predetermined location, including therapeutic applications and cell line engineering.

### CROSS-REFERENCE TO RELATED APPLICATIONS

This application claims priority to U.S. Provisional Applications 61/354,923, 61/382,773, and 61/484,005 which are hereby incorporated by reference in their entireties.

### BACKGROUND OF THE INVENTION

1. Field of the Invention

The present invention concerns a method for improving cleavage of DNA by rare-cutting endonucleases targeting specific DNA target sequences in loci of interest within genomes, the use of this method to design endonuclease variants with novel specificities for genome engineering, including therapeutic applications and cell line engineering.

2. Discussion of the Related Art

Since the first gene targeting experiments in yeast more than 25 years ago (Hinnen et al, 1978; Rothstein, 1983), homologous recombination (HR) has been used to insert, replace or delete genomic sequences in a variety of cells (Thomas and Capecchi, 1987; Capecchi et al, 2001; Smithies et al, 2001). HR is a very conserved DNA maintenance pathway involved in the repair of DNA double-strand breaks (DSBs) and other DNA lesions (Paques and Haber, 1999; Sung and Klein, 2006), but it also underlies many biological phenomenon, such as the meiotic reassortment of alleles in meiosis (Roeder et al, 1997). A competing pathway in DSBs repair events is the Non-Homologous End Joining (NHEJ) pathway which accounts for all DSBs repair events in the absence of an homologous repair matrix (Paques and Haber, 1999; van Gent et al, 2001). Although perfect re-ligation of the broken ends is probably the most frequent event, imperfect rejoining of the broken ends can result in the addition or deletion of one of several base pairs, inactivating the targeted open reading frame. Homologous gene targeting strategies have been used to knock out endogenous genes (Capecchi, M. R., Science, 1989, 244, 1288-1292, Smithies, O., Nature Medicine, 2001, 7, 1083-1086) or knock-in exogenous sequences in the chromosome. It can as well be used for gene correction, and in principle, for the correction of mutations linked with monogenic diseases. However, this application is in fact difficult, due to the low efficiency of the process (10⁻⁶ to 10⁻⁹ of transfected cells). The frequency of HR can be significantly increased by a specific DNA double-strand break (DSB) at a locus (Rouet et al, 1994; Choulika et al, 1995). Such DSBs can be induced by meganucleases, sequence-specific endonucleases that recognize large DNA recognition target sites (12 to 30 bp).

Meganucleases show high specificity to their DNA target, these proteins being able to cleave a unique chromosomal sequence and therefore do not affect global genome integrity. Natural meganucleases are essentially represented by homing endonucleases, a widespread class of proteins found in eukaryotes, bacteria and archae (Chevalier and Stoddard, 2001). Early studies of the I-SceI and HO homing endonucleases have illustrated how the cleavage activity of these proteins can be used to initiate HR events in living cells and have demonstrated the recombinogenic properties of chromosomal DSBs (Dujon et al, 1986; Haber, 1995). Since then, meganuclease-induced HR has been successfully used for genome engineering purposes in bacteria (Posfai et al, 1999), mammalian cells (Sargent et al, 1997; Donoho et al, 1998; Cohen-Tannoudji et al, 1998), mice (Gouble et al, 2006) and plants (Puchta et al, 1996; Siebert and Puchta, 2002).

Other specialized enzymes like integrases, recombinases, transposases and endonucleases have been proposed for site-specific genome modifications. For years, the use of these enzymes remained limited, due to the challenge of retargeting their natural specificities towards desired target sites. Indeed, the target sites of these proteins, or sequences with a sufficient degree of sequence identity, should be present in the sequences neighboring the mutations to be corrected, or within the gene to be inactivated, which is usually not the case, except in the case of pre-engineered sequences.

Meganucleases have emerged as scaffolds of choice for deriving genome engineering tools cutting a desired target sequence (Paques et al. Curr Gen Ther. 2007 7:49-66). Combinatorial assembly processes allowing to engineer meganucleases with modified specificities has been described by Arnould et al. J Mol Biol. 2006 355:443-458; Arnould et al. J Mol Biol. 2007 371:49-65; Smith et al. NAR 2006 34:e149; Grizot et al. NAR 2009 37:5405). Briefly, these processes rely on the identifications of locally engineered variants with a substrate specificity that differs from the substrate specificity of the wild-type meganuclease by only a few nucleotides.

Although these powerful tools are available, the functionality of the meganuclease on a particular target in a genome may also depend on the DNA target status such as accessibility, DNA modifications, as well as other features.

When interacting with DNA, all sequence-specific proteins form bonds with the individual bases of the target sequence (Saenger, 1983). Some of the bases in the target may be less important than others and sometimes all that is required is a consensus sequence to be present for binding to occur. In other situations the protein is completely specific in its requirements and will bind to only a single target sequence. Alteration in a base as substitution of methylcytosine for cytosine or methyladenine for adenine will affect binding or function of the protein.

DNA methylation is found almost ubiquitously in nature and the methyltransferases show evidence of a common evolutionary origin.

Physiological DNA methylation is accomplished by transfer of the methyl group from S-adenosyl methionine to 5 position of the pyrimidine ring of cytosine or the number 6 nitrogen of the adenine purine ring. DNA methylation is observed in most of the organisms at the different stages of evolution, in such a distinct species as E. coli and H. sapiens. However some species, like Drosophilae melanogaster lack DNA methylation [Bird, A., Tate, P., Nan, X., Campoy, J., Meehan, R., Cross, S., Tweedie, S., Charlton, J., and Macleod, D. (1995). Studies of DNA methylation in animals. J Cell Sci Suppl 19, 37-9.)

Extensive research on methylation was conducted on bacteria. In these lower forms, both adenine and cytosine can be methylated, and this modification is involved in DNA replication and arrangement. DNA methylation is catalyzed by a series of enzymes called DNA methyltransferases (DNA-MTases) which can catalyse cytosine or adenine methylation in different sequence context [Noyer-Weidner, M. and Trautner, T. A. (1993). Methylation of DNA in prokaryotes. EXS 64, 39-108.].

In Bacteria, adenine or cytosine methylations are mainly part of the restriction modification system, in which DNAs are methylated periodically throughout the genome. Foreign DNAs (which are not methylated in this manner) that are introduced into the cell are degraded by sequence-specific restriction enzymes which discriminate between endogenous and foreign DNA by its methylation pattern: Bacterial genomic DNA is not recognized by these restriction enzymes. The methylation of native DNA acts as a sort of primitive immune system, allowing the bacteria to protect themselves from infection by bacteriophage. These restriction enzymes are the basis of the modern Molecular Biology.

In addition, DNA methylation in prokaryotes is involved in the control of replication fidelity. During DNA replication the newly synthesised strand does not get methylated immediately, but analysed for mismatches by the mismatch repair system. When a mutation is found the correction takes place on the nonmethylated strand [Cooper, D. L., Lahue, R. S., and Modrich, P. (1993). Methyl-directed mismatch repair is bidirectional. J Biol Chem 268, 11823-9.].

In fungi, methylation vary both among species (levels of methylcytosine ranging from 0.5% to 5%) and among isolates of the same species (Thomas Binz, Nisha D'Mello, Paul A. Horgen (1998). “A Comparison of DNA Methylation Levels in Selected Isolates of Higher Fungi”. Mycologia 90 (5): 785-790). Although Saccharomyces and Schizosaccharomyces) have very little DNA methylation, the filamentous fungus Neurospora crassa has a well characterized methylation system (Eric U. Selker, Nikolaos A. Tountas, Sally H. Cross, Brian S. Margolin, Jonathan G. Murphy, Adrian P. Bird and Michael Freitag (2003). “The methylated component of the Neurospora crassa genome”. Nature 422 (6934): 893-897) that seems to be involved in state-specific control of gene expression.

In plants, methylation occurs mainly on the cytosine in CpG, CpNpG, and CpNpN context, where N represents any nucleotide but guanine. Methyltransferase enzymes, which transfer and covalently attach methyl groups onto DNA, are DRM2, MET1, and CMT3. Both the DRM2 and MET1 proteins share significant homology to the mammalian methyltransferases DNMT3 and DNMT1, respectively, whereas the CMT3 protein is unique to the plant kingdom.

In mammals, DNA methylation occurs mainly at the C5 position of CpG dinucleotides (cytosine-phosphate-guanine sites; that is, where a cytosine is directly followed by a guanine in the DNA sequence) and accounts for about 1% of total DNA bases. It is carried out by two general classes of enzymatic activities—maintenance methylation and de novo methylation. The bulk of mammalian DNA has about 40% to 90% of CpG sites methylated (Tucker K L (June 2001). “Methylated cytosine and the brain: a new base for neuroscience”. Neuron 30 (3): 649-52). This average pattern conceals intriguing temporal and spatial variation. During a discrete phase of early mouse development, methylation levels in the mouse decline sharply to about 30% of the typical somatic level (Monk et al, 1987; Kafri et al, 1992). The most striking feature of vertebrate DNA methylation patterns is the presence of clusters in certain areas, known as CpG islands which are GC rich (made up of about 65% CG residue) that is, unmethylated GC-rich regions that possess high relative densities of CpG. These CpG islands, which represent 1-2% of the human genome, are present in the 5′ regulatory regions of many mammalian genes (for review, see Bird et al, 1987).

These processes are essential for normal development and are associated with a number of key processes including genomic imprinting, X-chromosome inactivation, suppression of repetitive elements and carcinogenesis.

In early mammalian development, the genome within the germ cells is demethylated, while chromosomes in the remaining cells retain the parental methylation patterns. De novo methylation of the germ cells occurs, modifying and adding epigenetic information to the genome based on the sex of the individual [Carroll, Sean B.; Wessler, Susan R.; Griffiths, Anthony J. F.; Lewontin, Richard C. (2008). Introduction to genetic analysis (9th ed.). New York: W.H. Freeman and CO. p. 403. ISBN 0-7167-6887-9.]. By blastula stage, the methylation is complete. This process is referred to as “epigenetic reprogramming” (Mann M R, Bartolomei M S (2002). “Epigenetic reprogramming in the mammalian embryo: struggle of the clones”. Genome Biol. 3 (2): 1003.1-.4.). Increasing evidence is revealing a role of methylation in the interaction of environmental factors with genetic expression. Differences in maternal care during the first 6 days of life in the rat induce differential methylation patterns in some promoter regions and thus influencing gene expression. (Weaver I C (2007). “Epigenetic programming by maternal behavior and pharmacological intervention. Nature versus nurture: let's call the whole thing off”. Epigenetics 2 (1): 22-8.). In cancer, the dynamics of genetic and epigenetic gene silencing are very different. CpG sites are hotspots for mutation in the human germline [Cooper, D. N. and Youssoufian, H. (1988). The CpG dinucleotide and human genetic disease. Hum Genet 78, 151-5]. More recently it has become clear that they can be also hotspots for inactivating mutations in tumour suppresser genes [Rideout, W. M. 3., Coetzee, G. A., Olumi, A. F., and Jones, P. A. (1990). 5-Methylcytosine as an endogenous mutagen in the human LDL receptor and p53 genes. Science 249, 1288-90. Fearon, E. R. and Jones, P. A. (1992). Progressing toward a molecular description of colorectal cancer development. FASEB J 6, 2783-90]. About 25% of all mutations in p53 gene in all human cancers studied occur at CpG sites, and almost 50% occur at methylation sites in colon cancer [Greenblatt, M. S., Bennett, W. P., Hollstein, M., and Harris, C. C. (1994). Mutations in the p53 tumor suppressor gene: clues to cancer etiology and molecular pathogenesis. Cancer Res 54, 4855-78.]. More than a decade ago it was shown that global genomic levels of DNA methylation are lower in cancer cells than in normal tissue [Lapeyre, J. N. and Becker, F. F. (1979). 5-Methylcytosine content of nuclear DNA during chemical hepatocarcinogenesis and in carcinomas which result. Biochem Biophys Res Commun 87, 698-705. Gama-Sosa, M. A., Slagel, V. A., Trewyn, R. W., Oxenhandler, R., Kuo, K. C., Gehrke, C. W., and Ehrlich, M. (1983). The 5-methylcytosine content of DNA from human tumors. Nucleic Acids Res 11, 6883-94. Feinberg, A. P., Gehrke, C. W., Kuo, K. C., and Ehrlich, M. (1988). Reduced genomic 5-methylcytosine content in human colonic neoplasia. Cancer Res 48, 1159-61). Despite the clear association of DNA hypomethylation with both spontaneous and experimentally derived tumours, the exact role of this change is poorly understood. The same tumour cells which were described to have the overall genomic hypomethylation frequently have regions of dense hypermethylation. The fact that most of nonmethylated cytosines are located within CpG islands suggests that the normally nonmethylated CpG islands within 5′ regulatory regions are the primary targets for aberrant hypermethylation in tumour cells [Bird, A. P. (1995). Gene number, noise reduction and biological complexity. Trends Genet 11, 94-100].

In addition, the protective function of DNA methylation is similar in eukaryotes and prokaryotes. In humans and rodents inserted viral sequences can become methylated in association with silencing of the introduced genes [Kisseljova, N. P., Zueva, E. S., Pevzner, V. S., Grachev, A. N., and Kisseljov, F. L. (1998). De novo methylation of selective CpG dinucleotide clusters in transformed cells mediated by an activated N-ras. Int J Oncol 12, 203-9]. The same mechanism is involved in silencing of transgenes in mice [Sasaki, H., Allen, N. D., and Surani, M. A. (1993). DNA methylation and genomic imprinting in mammals. EXS 64, 469-86. Collick, A., Reik, W., Barton, S. C., and Surani, A. H. (1988). CpG methylation of an X-linked transgene is determined by somatic events postfertilization and not germline imprinting. Development 104, 235-44]. Thus function of DNA methylation machinery for recognition and/or eliminating of foreign DNA seem to be conserved in evolution.

DNA methylation is very important for gene expression and regulation in eukaryotes. For example, cell differentiation is regulated by DNA methylation at gene transcriptional level. Moreover, many results show that DNA conformation may be effected by DNA methylation. As a result the interaction between the upstream regulating region of gene and some protein factors related gene transcription is changed in time and space. However, restriction enzymes provide the clearest example where methylation of DNA prevents its cleavage by interfering with the binding and/or function of the nuclease. Some or all of the sites for a restriction endonuclease may be resistant to cleavage when isolated from strains expressing the Dam or Dcm methylases if the methylase recognition site overlaps the endonuclease recognition site. For example, plasmid DNA isolated from dam⁺ E. coli is completely resistant to cleavage by MboI, which cleaves at GATC sites. The type II enzymes which act as dimers with one subunit cleaving each strand on the DNA, is blocked by methylation of only one strand. The type I restriction enzymes are also affected by DNA methylation. For the cleavage occurs, two molecules need to bind to the target, the enzyme bound at the recognition sequence translocates DNA toward itself; and when translocation causes neighboring enzymes to meet, they cut the DNA between them. (Model for how type I restriction enzymes select cleavage sites in DNA. Studier F W, Bandyopadhyay P K. Proc Natl Acad Sci USA. 1988 July; 85(13):4677-81.). If the DNA is hemimethylated, the enzyme will leaves the DNA, so DNA translocation can not occur. These controlled reactions involve complex changes in the nature of the DNA-protein complex (Bickle T A (1982) Cold Spring Harbor Monogr. Ser. 14, 85-108).

Moreover, restriction enzymes with the same specificity towards a particular DNA target (so called isoschizomers) may behave differently on regards of DNA methylation of the target. In some cases, only one out of a isoschizomers family can recognize both the methylated as well as unmethylated forms of restriction sites. In contrast, the other restriction enzyme can recognize only the unmethylated form of the restriction site. For example, the restriction enzymes HpaII & MspI are isoschizomers, as they both recognize the sequence 5′-CCGG-3′ when it is unmethylated. But when the second C of the sequence is methylated, only MspI can recognize both the forms while HpaII cannot.

The inventors have now found that CpG content of a DNA sequence and the level of methylation of such CpG nucleotides have an influence on the cleavage activity of rare-cutting endonucleases such as meganucleases. For the first time, inventors have shown that the cleavage activity of rare-cutting endonuclease, sensitive to methylation, is dependent on the locations of CpG motifs within said DNA sequence.

### BRIEF SUMMARY OF THE INVENTION

The present invention concerns novel methods for improving cleavage of DNA by rare-cutting endonucleases, overcoming DNA modification constraints, particularly DNA methylation, thereby giving new tools for genome engineering, particularly to increase the integration efficiency of a transgene into a genome at a predetermined location, including therapeutic applications and cell line engineering. While the above objects highlight certain aspects of the invention, additional objects, aspects and embodiments of the invention are found in the following detailed description of the invention. In addition to the preceding features, the invention further comprises other features which will emerge from the description which follows. The description refers to examples illustrating the use of I-CreI meganuclease variants according to the invention, as well as to the appended drawings. A more complete appreciation of the invention and many of the attendant advantages thereof will be readily obtained as the same becomes better understood by reference to the following figures in conjunction with the detailed description below.

### BRIEF DESCRIPTION OF THE DRAWINGS

FIG. 1: Spectrofluorimetric titration of fluorescein-labeled C1221 by I-CreI. 50 nM C1221, containing either 0 or 4 methylated CG on both strands, was incubated with increasing concentrations of I-CreI (from 0 to 1.5 μM) in binding buffer (10 mM Tris-HCl, 150 mM NaCl, 10 mM CaCl₂, 10 mM DTT, pH8) at 25° C. After 30 minutes incubation, the fluorescence anisotropy of the mixture was recorded and then plotted as a function of I-CreI concentration.

FIG. 2: In vitro cleavage of unmethylated or methylated C1221 by I-CreI. A constant amount of C1221 (50 nM) was incubated with increasing concentrations of I-CreI (0 to 2 μM) in reaction buffer (10 mM Tris-HCl, 150 mM NaCl, 10 mM MgCl₂, pH8) for an hour at 37° C. The reaction was stopped and the remaining uncleaved C1221 was quantify and plotted as function of I-CreI concentration.

FIG. 3: In vitro cleavage of unmethylated or methylated C1221 by I-CreI D75N. 50 nM C1221 containing either 0, 1, 2 or 3 methylated CG was incubated with increasing concentrations of I-CreI D75N (0 to 2 μM) in reaction buffer (10 mM Tris-HCl, 150 mM NaCl, 10 mM MgCl₂, pH8) for an hour at 37° C. The reaction was stopped and cleaved and uncleaved C1221 (top and bottom panel respectively) were quantify and plotted as function of I-CreI D75N concentration.

FIG. 4: Spectrofluorimetric titration of fluorescein-labeled C1234 by I-Cre I wild type. 25 nM of C1234 duplex was incubated with increasing concentrations of I-Cre I [from 0 to 400 nM, only 0-80 nM shown] in binding buffer (10 mM Tris-HCl, 400 mM NaCl, 10 mM CaCl₂, 10 mM DTT, pH8) at 25° C. After 30 minutes incubation, the fluorescence anisotropy of the mixture was recorded with a Pherastar Plus (BMG Labtech) operating in fluorescence polarization end point mode with excitation and emission wavelengths set to 495 and 520 nm respectively. Normalized fluorescence anisotropy is plotted as a function of active I-Cre I concentration. Apparent dissociation constant were determined by fitting raw data by an hyperbolic function (Aₓ=(A_{∞}*[Meganuclease])/(K_{d}+[Meganuclease]) with Aₓ, the fluorescence anisotropy value obtained at a given meganuclease concentration, A_{∞}, the fluorescence anisotropy value obtained at saturating concentration of meganuclease and K_{d} the dissociation constant of the equilibrium studied).

FIG. 5: In vitro cleavage of unmethylated or methylated C1234 by I-Cre I wild type. A constant amount of C 1234 duplex (50 nM) was incubated with an excess of I-Cre I (1.5 μM, final concentration) in the reaction buffer (10 mM Tris-HCl, 150 mM NaCl, pH8) at 37° C. Cleavage reaction was triggered by the addition of MgCl₂ and then stopped after different time lengths by the addition of the stop buffer. This was followed by one hour of incubation at 37° C. to digest I-Cre I and release free DNA molecules. Cleaved and uncleaved DNA products were separated by PAGE using a TGX Any kD precast gel (Bio-Rad), stained with SYBR Green and then quantified using Quantity One software (Bio-Rad). Disappearance of substrate (uncleaved DNA) is plotted as a function of time.

FIG. 6: Model of I-Cre I:C1234_Me full complex based on I-Cre I:C1234 crystal structure (ref PDB). I-Cre I:C1234 crystal structure was used to model I-Cre I:C1234_Me full using Pymol software. A: overall structure model of I-Cre I:C1234_Me full. C1234 “a” and “b” strands are displayed in cyan and magenta respectively and the polypeptide chain is displayed in wheat. B: close up of the steric clash between cytosin-2b and Valine 73. C: close up of the steric clash between Cytosine-5b and Isoleucine 24. D: close up of cytosine-3a and the two nearest amino acids Arginine 70, Glycine 71. E: close up of cytosine-6a and the two nearest amino acids Tyrosine 66 and Arginine 68.

FIG. 7: Spectrofluorimetric titration of fluorescein-labeled XPC4.1 by XPC4. 50 nM of XPC4.1 duplex was incubated with increasing concentrations of XPC4 (from 0 to 800M) in binding buffer (10 mM Tris-HCl, 150 mM NaCl, 10 mM CaCl₂, 10 mM DTT, pH8) at 25° C. After 30 minutes incubation, the fluorescence anisotropy of the mixture was recorded with a Pherastar Plus (BMG Labtech) operating in fluorescence polarization end point mode with excitation and emission wavelengths set to 495 and 520 nm respectively. Normalized fluorescence anisotropy is plotted as a function of XPC4 concentration. Apparent dissociation constant were determined by fitting raw data by an hyperbolic function (Aₓ=(A_{∞}*[Meganuclease])/(K_{d}+[Meganuclease]) with Aₓ, the fluorescence anisotropy value obtained at a given meganuclease concentration, A_{∞} the fluorescence anisotropy value obtained at saturating concentration of meganuclease and K_{d} the dissociation constant of the equilibrium studied).

FIG. 8: In vitro cleavage of unmethylated or methylated XPC4.1 by XPC4. A constant amount of XPC4.1 duplex (50 nM) was incubated with an excess of XPC4 (1.5 μM, final concentration) in the reaction buffer (10 mM Tris-HCl, 150 mM NaCl, pH8) at 37° C. Cleavage reaction was triggered by the addition of MgCl₂ and then stopped after different time lengths by the addition of the stop buffer. This was followed by one hour of incubation at 37° C. to digest XPC4 and release free DNA molecules. Cleaved and uncleaved DNA products were separated by PAGE using a TGX Any kD precast gel (Bio-Rad), stained with SYBR Green and then quantified using Quantity One software (Bio-Rad). Disappearance of substrate (uncleaved DNA) is plotted as a function of time.

FIG. 9: Chromatograms of sequencing reactions at the XPC4 target locus, made after bisulfite treatment. Cells were pre-treated with 5-aza-2-deoxycytidine at 0.2 μM or 1 μM 48 hours before transfection with the XPC4 meganuclease or with an empty vector. The treatment was maintained 48 hours post-transfection. As a control, we used cells not treated with 5-aza-2-deoxycytidine (NT). Two days post transfection, genomic DNA was extracted and treated with bisulfite, which converts cytosine, but not 5-methylcytosine into uracil. DNA from the XPC4 target locus region was amplified by PCR, and sequenced. Sequence of the XPC4 target is indicated on top (XPC4 target), with the two CpG motives being underlined. On the chromatograms, 5-methyl-cytosines appear as cytosines (C), and non methylated cytosines (converted to uracil) as thymines (T). In the presence of 5-aza-2-deoxycytidine, a fraction of the CpG motives is demethylated, resulting in a dual C/T peak.

FIG. 10: Frequencies of mutagenesis events measured by deep sequencing. Cells were pre-treated with 5-aza-2-deoxycytidine at 0.2 μM or 1 μM 48 hours before transfection with XPC4 meganuclease or empty vector. The treatment was maintained 48 hours post-transfection. Two days post-transfection, the genomic DNA was extracted and a PCR with primers surrounding target site was performed. The results were expressed as a percentage of PCR fragments containing a mutation.

FIG. 11: XPC4 meganuclease efficiency is impaired by DNA methylation in vivo. 293H cells were co-transfected with 3 μg of XPC4 meganuclease expressing vector or empty vector and 2 μg of DNA repair matrix vector in presence or absence of DNA methylation inhibitor (5-aza-2′deoxycytidine). 480 individual cellular clones were analyzed in each condition for the presence of targeted events using specific PCR amplification.

FIG. 12: Spectrofluorimetric titration of fluorescein-labeled ADCY9.1 by ADCY9. 50 nM of ADCY9.1 duplex was incubated with increasing concentrations of ADCY9 (from 0 to 1.5 μM) in binding buffer (10 mM Tris-HCl, 150 mM NaCl, 10 mM CaCl₂, 10 mM DTT, pH8) at 25° C. After 30 minutes incubation, the fluorescence anisotropy of the mixture was recorded with a Pherastar Plus (BMG Labtech) operating in fluorescence polarization end point mode with excitation and emission wavelengths set to 495 and 520 nm respectively. Normalized fluorescence anisotropy is plotted as a function of ADCY9 concentration. Apparent dissociation constant were determined by fitting raw data by an hyperbolic function (Aₓ=(A_{∞}*[Meganuclease])/(K_{d}+[Meganuclease]) with Aₓ, the fluorescence anisotropy value obtained at a given meganuclease concentration, A_{∞} the fluorescence anisotropy value obtained at saturating concentration of meganuclease and K_{d} the dissociation constant of the equilibrium studied).

FIG. 13: In vitro cleavage of unmethylated or methylated ADCY9.1 by ADCY9. A constant amount of ADCY9.1 duplex (50 nM) was incubated with an excess of ADCY9 (1.5 μM, final concentration) in the reaction buffer (10 mM Tris-HCl, 150 mM NaCl, pH8) at 37° C. Cleavage reaction was triggered by the addition of MgCl₂ and then stopped after different time lengths by the addition of the stop buffer. This was followed by one hour of incubation at 37° C. to digest ADCY9 and release free DNA molecules. Cleaved and uncleaved DNA products were separated by PAGE using a TGX Any kD precast gel (Bio-Rad), stained with SYBR Green and then quantified using Quantity One software (Bio-Rad). Disappearance of substrate (uncleaved DNA) is plotted as a function of time.

FIG. 14: ADCY9 meganuclease efficiency is impaired by DNA methylation in vivo. 293H cells were co-transfected with 5 μg of XPC4 meganuclease expressing vector or empty vector and 2 μg of DNA repair matrix vector in presence or absence of DNA methylation inhibitor (5-aza-2′deoxycytidine). 480 individual cellular clones were analyzed in each condition for the presence of targeted events using specific PCR amplification.

FIG. 15: Chromatogram. In order to determine the methylation status of the two CpG motives present in the XPC4 target sequence, genomic DNA was extracted, and treated with bisulfite. Bisulfite treatment is based on a chemical reaction of sodium bisulfite with DNA that converts unmethylated cytosines into uracil whereas methylated cytosines remain unchanged. DNA was then amplified by PCR and sequenced. Examples of sequences are shown in FIG. 15. In presence of si_AS, no cytosine conversion was observed in XPC4 target sequence, showing that both CpG were methylated in the vast majority of the cells. After transfection with si_DNMT1, dual peaks were observed in the chromatogram, showing that in the treated cell population, the two CpG could be methylated or unmethylated. For one of the two CpG (TCGAGATGTCACACAGAGGTACGA; SEQ ID NO: 24) the amount of unmethylated C was estimated to 20 and 30% of total after 1 nM and 5 nM of si_DNMT1, respectively. For the other CpG (TCGAGATGTCACACAGAGGTACGA; SEQ ID NO: 24) the amount of unmethylated C was estimated to 25 and 50% after 1 nM and 5 nM of si_DNMT1, respectively.

### DETAILED DESCRIPTION OF THE INVENTION

Unless specifically defined herein below, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986). The methods disclosed below preferably comprise each of the recited steps, though they may be performed using one or more of the recited steps independently or in conjunction with other steps.

According to a first aspect of the present invention is a method for improving cleavage of DNA from a chromosomal locus in a cell by an engineered rare-cutting endonuclease sensitive to methylation, comprising the steps of:
(i) identifying at said chromosomal locus a cleavable DNA target sequence devoided of any CpG sequence;
(ii) engineering said rare-cutting endonuclease;
(iii) contacting said DNA target sequence with said rare-cutting endonuclease.

In a preferred embodiment of this first aspect said engineered rare-cutting endonuclease sensitive to methylation is a meganuclease. In a more preferred embodiment said engineered rare-cutting endonuclease sensitive to methylation is a meganuclease from the LAGLIDADG family. In another more preferred embodiment, said engineered rare-cutting endonuclease sensitive to methylation is a meganuclease derived from the I-CreI meganuclease.

According to a second aspect of the present invention is a method for improving cleavage of DNA from a chromosomal locus in a cell by an engineered rare-cutting endonuclease sensitive to methylation, comprising the steps of:
(i) identifying at said chromosomal locus a DNA target sequence of more than 14 base pairs (bp) in length wherein said DNA target sequence contains no more than 3 CpG motifs;
(ii) engineering said rare-cutting endonuclease;
(iii) contacting said DNA target sequence with said rare-cutting endonuclease.

In a preferred embodiment, said DNA target sequence is 22-24 base pairs (bp) in length. In a preferred embodiment of this second aspect said engineered rare-cutting endonuclease sensitive to methylation is a meganuclease. In a more preferred embodiment said engineered rare-cutting endonuclease sensitive to methylation is a meganuclease from the LAGLIDADG family. In another more preferred embodiment, said engineered rare-cutting endonuclease sensitive to methylation is a meganuclease derived from the I-CreI meganuclease.

In another preferred embodiment of this second aspect said DNA target sequence contains no CpG motif in position −2 to +2. In a more preferred embodiment, said DNA target sequence contains no CpG motif neither in position ±5 to ±3 nor in position −2 to +2. In another preferred embodiment, said DNA target sequence contains no more than two CpG dinucleotides. In a more preferred embodiment, said DNA target sequence contains no more than one CpG dinucleotide. In a more preferred embodiment, said DNA target sequence contains no CpG dinucleotide.

In another preferred embodiment of this second aspect of the present invention, said cell is a eukaryotic cell. In a more preferred embodiment, said cell is a plant cell. In another more preferred embodiment, said cell is a mammalian cell.

According to a third aspect of the present invention is a method to improve cleavage of DNA from a chromosomal locus in a chosen cell type or organism, by an engineered rare-cutting endonuclease sensitive to methylation, comprising the following steps:
(i) determining CpG content of potential DNA target sequences;
(ii) determining methylation level of said DNA target sequences in at least one cell type related to the chosen cell type or organism;
(iii) selecting potential DNA target sequences displaying no methylation;
(iv) engineering said rare-cutting endonuclease;
(v) contacting said DNA target sequence with said rare-cutting endonuclease.

In a preferred embodiment of this third aspect said engineered rare-cutting endonuclease sensitive to methylation is a meganuclease. In a more preferred embodiment said engineered rare-cutting endonuclease sensitive to methylation is a meganuclease from the LAGLIDADG family. In another more preferred embodiment, said engineered rare-cutting endonuclease sensitive to methylation is a meganuclease derived from the I-CreI meganuclease.

In this aspect of the invention, the bisulfite method can be used to identify specific methylation patterns within the considered sample. It consists of treating DNA with bisulfite, which causes unmethylated cytosines to be converted into uracil while methylated cytosines remain unchanged (Shapiro et al., 1973). The DNA is then amplified by PCR with specific primers designed on bisulfite converted template. The methylation profile of the bisulfite treated DNA is determined by DNA sequencing (Frommer et al., 1992). The methylation status can also simply inferred from the literature or from public databases, for example when the specific target sequence belongs to a known unmethylated CpG island.

In another preferred embodiment of this aspect of the invention, the methylation level is assayed in the cell type of interest. In a more preferred embodiment, said cell is a eukaryotic cell. In a more particularly preferred embodiment, said cell is a plant cell. In another more preferred embodiment, said cell is a mammalian cell. In another more preferred embodiment, the potential target sites displaying no methylation are GC-rich regions such as unmethylated GC-rich regions that possess high relative densities of CpG, known as CpG islands.

According to a fourth aspect of the invention, is a method to select a target cell type for a rare-cutting endonuclease, said rare-cutting endonuclease cleaving a DNA target sequence comprising at least one CpG dinucleotide, comprising the following steps:
(i) determining methylation level of said DNA target sequence in several cell types;
(ii) selecting cell type displaying no methylation;
(iii) contacting said DNA target sequence with said rare-cutting endonuclease.

In a preferred embodiment of this fourth aspect said engineered rare-cutting endonuclease sensitive to methylation is a meganuclease. In a more preferred embodiment said engineered rare-cutting endonuclease sensitive to methylation is a meganuclease from the LAGLIDADG family. In another more preferred embodiment, said engineered rare-cutting endonuclease sensitive to methylation is a meganuclease derived from the I-CreI meganuclease.

In another preferred embodiment of this aspect of the invention, said cell is a eukaryotic cell. In a more particularly preferred embodiment, said cell is a plant cell. In another more preferred embodiment, said cell is a mammalian cell. In a preferred embodiment, the potential target sites displaying no methylation are GC-rich regions such as unmethylated GC-rich regions that possess high relative densities of CpG, known as CpG islands.

According to a fifth aspect of the invention, is a method to improve cleavage of a chromosomal DNA target sequence comprising at least one methylated CpG dinucleotide, by an engineered or natural rare-cutting endonuclease, sensitive to methylation comprising the following steps:
(i) treating the target cell with an agent inhibiting methylation;
(ii) contacting said DNA target sequence with said engineered or natural rare-cutting endonuclease, sensitive to methylation.

In a preferred embodiment of this fifth aspect said engineered rare-cutting endonuclease sensitive to methylation is a meganuclease. In a more preferred embodiment said engineered rare-cutting endonuclease sensitive to methylation is a meganuclease from the LAGLIDADG family. In another more preferred embodiment, said engineered rare-cutting endonuclease sensitive to methylation is a meganuclease derived from the I-CreI meganuclease.

In this aspect of the present invention, said demethylating agent is selected from the group comprising DNA Methyltransferase inhibitor.

In biochemistry, the DNA methyltransferase (DNMT) family of enzymes catalyze the transfer of a methyl group to DNA. Three active DNA methyltransferases have been identified in mammals. DNMT1 is the most abundant DNMT in mammalian cells, and considered to be the key maintenance methyltransferase in mammals. The process of cytosine methylation is reversible and may be altered by biochemical and biological manipulations. Currently available, nucleoside-based DNMT inhibitors such as 5-azacytidine, 5-aza-2′deoxycytidine, zebularine, are analogues of cytosine (Cheng et al., Cancer cell, 2004; Momparler., Sem Hematol, 2005; Zhou et al., JMB 2002). They are incorporated into DNA during replication forming covalent adducts with cellular DNMT, thereby depleting its enzyme activity and leading to demethylation of genomic DNA. Making reference to their action or the consequence of their action, these agents are “agent inhibiting methylation” or “demethylating agents”. Thus, incubation of the cells with DNMT inhibitor leads to a state of unmethylated DNA.

In preferred embodiment of this aspect of the invention, said cell is a eukaryotic cell. In a more particularly preferred embodiment, said cell is a plant cell. In another more preferred embodiment, said cell is a mammalian cell.

Products made or identified by the methods disclosed herein or those used to practice these methods are also disclosed herein.

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased/improved cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
By “CpG” or “CpG motif” or “CpG content” or “CpG sequence” is intended CpG dinucleotides, that is Cytosine-phosphate-Guanine dinucleotides where a cytosine is directly followed by a guanine in the DNA sequence. By “CpG islands” is intended clusters in certain areas of mammalian genomes, which are GC-rich regions (made up of about 65% CG residue), unmethylated and that possess high relative densities of CpG. These CpG islands, which represent 1-2% of the human genome, are present in the 5′ regulatory regions of many mammalian genes (for review, see Bird et al, 1987).
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “related to”, particularly in the expression “one cell type related to the chosen cell type or organism”, is intended a cell type or an organism sharing characteristics with said chosen cell type or said chosen organism; this cell type or organism related to the chosen cell type or organism, can be derived from said chosen cell type or organism or not.
by “subdomain” it is intended the region of a LAGLIDADG homing endonuclease core domain which interacts with a distinct part of a homing endonuclease DNA target half-site.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the targeted gene comprised in the locus of interest and the repair matrix, wherein the genomic sequence containing the DNA target is replaced by the third portion of the repair matrix and a variable part of the first and second portions of the repair matrix.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of pdb accession code 1g9y, corresponding to the sequence SEQ ID NO: 1 in the sequence listing.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 23), also called C1221.
by “domain” or “core domain” is intended the “LAGLIDADG homing endonuclease core domain” which is the characteristic αββαββα fold of the homing endonucleases of the LAGLIDADG family, corresponding to a sequence of about one hundred amino acid residues. Said domain comprises four beta-strands (β₁β₂β₃β₄) folded in an anti-parallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “subdomain” is intended the region of a LAGLIDADG homing endonuclease core domain which interacts with a distinct part of a homing endonuclease DNA target half-site.
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).
by “beta-hairpin” is intended two consecutive beta-strands of the antiparallel beta-sheet of a LAGLIDADG homing endonuclease core domain (β₁β₂ or β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”, “site of interest”, “recognition site”, “polynucleotide recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicate above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of different halves of two parent meganuclease target sequences. In addition at least one half of said target may comprise the combination of nucleotides which are bound by at least two separate subdomains (combined DNA target).
The term “endonuclease” refers to any wild-type or variant enzyme capable of catalyzing the hydrolysis (cleavage) of bonds between nucleic acids within of a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 12 base pairs (bp) in length, more preferably of 14-45 bp. Rare-cutting endonucleases significantly increase HR by inducing DNA double-strand breaks (DSBs) at a defined locus (Rouet et al, 1994; Choulika et al, 1995; Pingoud et Silva, 2007). Rare-cutting endonucleases can for example be a homing endonuclease (Paques et al. Curr Gen Ther. 2007 7:49-66), a chimeric Zinc-Finger nuclease (ZFN) resulting from the fusion of engineered zinc-finger domains with the catalytic domain of a restriction enzyme such as FokI (Porteus et al. Nat Biotechnol. 2005 23:967-973) or a chemical endonuclease (Arimondo et al. Mol Cell Biol. 2006 26:324-333; Simon et al. NAR 2008 36:3531-3538; Eisenschmidt et al. NAR 2005 33:7039-7047). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer Ann NY Acad Sci 2005 1058: 151-61). Such chemical endonucleases are comprised in the term “endonuclease” according to the present invention. In the scope of the present invention is also intended any fusion between molecules able to bind DNA specific sequences and agent/reagent/chemical able to cleave DNA or interfere with cellular proteins implicated in the DSB repair (Majumdar et al. J. Biol. Chem 2008 283, 17:11244-11252; Liu et al. NAR 2009 37:6378-6388); as a non limiting example such a fusion can be constituted by a specific DNA-sequence binding domain linked to a chemical inhibitor known to inhibate re-ligation activity of a topoisomerase after DSB cleavage.

Rare-cutting endonucleases can also be for example TALENs, a new class of chimeric nucleases using a FokI catalytic domain and a DNA binding domain derived from Transcription Activator Like Effector (TALE), a family of proteins used in the infection process by plant pathogens of the Xanthomonas genus (Boch, Scholze et al. 2009; Moscou and Bogdanove 2009; Christian, Cermak et al. 2010; Li, Huang et al. 2011). The functional layout of a FokI-based TALE-nuclease (TALEN) is essentially that of a ZFN, with the Zinc-finger DNA binding domain being replaced by the TALE domain. As such, DNA cleavage by a TALEN requires two DNA recognition regions flanking an unspecific central region. Rare-cutting endonucleases encompassed in the present invention can also be derived from TALENs.

Rare-cutting endonuclease can be a homing endonuclease, also known under the name of meganuclease. Such homing endonucleases are well-known to the art (see e.g. Stoddard, Quarterly Reviews of Biophysics, 2006, 38:49-95). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease.

In the wild, meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffolds to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, and thus dimerize to cleave palindromic or pseudo-palindromic target sequences.

Although the LAGLIDADG peptide is the only conserved region among members of the family, these proteins share a very similar architecture. The catalytic core is flanked by two DNA-binding domains with a perfect two-fold symmetry for homodimers such as I-CreI (Chevalier, et al., Nat. Struct. Biol., 2001, 8, 312-316), I-MsoI (Chevalier et al., J. Mol. Biol., 2003, 329, 253-269) and I-CeuI (Spiegel et al., Structure, 2006, 14, 869-880) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-Anil (Bolduc et al., Genes Dev., 2003, 17, 2875-2888). Both monomers and both domains (for monomeric proteins) contribute to the catalytic core, organized around divalent cations. Just above the catalytic core, the two LAGLIDADG peptides also play an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), whose protein splicing domain is also involved in DNA binding.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Application WO 03/078619 (Cellectis) and WO 2004/031346 (Fred Hutchinson Cancer Research Center, Stoddard et al)) have demonstrated the plasticity of LAGLIDADG proteins.

Different groups have also used a semi-rational approach to locally alter the specificity of the I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156 (Cellectis); Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and 177 of I-CreI were mutagenized and a collection of variants with altered specificity at positions ±3 to 5 of the DNA target (5NNN DNA target) were identified by screening (International PCT Applications WO 2006/097784 and WO 2006/097853 (Cellectis); Arnould et al., J. Mol. Biol., 2006, 355, 443-458; Smith et al., Nucleic Acids Res., 2006, 34, e149).
Residues K28, N30 and Q38 or N30, Y33 and Q38 or K28, Y33, Q38 and S40 of I-CreI were mutagenized and a collection of variants with altered specificity at positions ±8 to 10 of the DNA target (10NNN DNA target) were identified by screening (Smith et al., Nucleic Acids Res., 2006, 34, e149; International PCT Applications WO 2007/060495 and WO 2007/049156 (Cellectis)).

Two different variants were combined and assembled in a functional heterodimeric endonuclease able to cleave a chimeric target resulting from the fusion of two different halves of each variant DNA target sequence (Arnould et al., precited; International PCT Applications WO 2006/097854 and WO 2007/034262).

Furthermore, residues 28 to 40 and 44 to 77 of I-CreI were shown to form two partially separable functional subdomains, able to bind distinct parts of a homing endonuclease target half-site (Smith et al. Nucleic Acids Res., 2006, 34, e149; International PCT Applications WO 2007/049095 and WO 2007/057781 (Cellectis)).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/049095 and WO 2007/057781 (Cellectis)).

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

The combination of the two former steps allows a larger combinatorial approach, involving four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest. The assembly of four sets of mutations into heterodimeric endonucleases cleaving a model target sequence or a sequence from different genes has been described in the following Cellectis International patent applications: XPC gene (WO2007/093918), RAG gene (WO2008/010093), HPRT gene (WO2008/059382), beta-2 microglobulin gene (WO2008/102274), Rosa26 gene (WO2008/152523), Human hemoglobin beta gene (WO2009/13622) and Human interleukin-2 receptor gamma chain gene (WO2009019614).

These variants can be used to cleave genuine chromosomal sequences and have paved the way for novel perspectives in several fields, including gene therapy.

Examples of such endonuclease include I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I, I-MsoI.

A homing endonuclease can be a LAGLIDADG endonuclease such as I-SceI, I-CreI, I-CeuI, I-MsoI, and I-DmoI.

Said LAGLIDADG endonuclease can be I-Sce I, a member of the family that contains two LAGLIDADG motifs and functions as a monomer, its molecular mass being approximately twice the mass of other family members like I-CreI which contains only one LAGLIDADG motif and functions as homodimers.

Endonucleases mentioned in the present application encompass both wild-type (naturally-occurring) and variant endonucleases. Endonucleases according to the invention can be a “variant” endonuclease, i.e. an endonuclease that does not naturally exist in nature and that is obtained by genetic engineering or by random mutagenesis, i.e. an engineered endonuclease. This variant endonuclease can for example be obtained by substitution of at least one residue in the amino acid sequence of a wild-type, naturally-occurring, endonuclease with a different amino acid. Said substitution(s) can for example be introduced by site-directed mutagenesis and/or by random mutagenesis. In the frame of the present invention, such variant endonucleases remain functional, i.e. they retain the capacity of recognizing and specifically cleaving a target sequence to initiate gene targeting process.

The variant endonuclease according to the invention cleaves a target sequence that is different from the target sequence of the corresponding wild-type endonuclease. Methods for obtaining such variant endonucleases with novel specificities are well-known in the art.

Endonucleases variants may be homodimers (meganuclease comprising two identical monomers) or heterodimers (meganuclease comprising two non-identical monomers).

Endonucleases with novel specificities can be used in the method according to the present invention for gene targeting and thereby integrating a transgene of interest into a genome at a predetermined location.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step (a) or the meganuclease from which the second series of variants are derived in step (b), or the meganuclease from which the third series of variants are derived in step (k).
by “vector” is intended a nucleic acid molecule capable of transporting another nucleic acid to which it has been linked.
by “homologous” is intended a sequence with enough identity to another one to lead to homologous recombination between sequences, more particularly having at least 95% identity, preferably 97% identity and more preferably 99% or any intermediate value or subrange.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
By a “TALE-nuclease” (TALEN) is intended a fusion protein consisting of a DNA-binding domain derived from a Transcription Activator Like Effector (TALE) and one FokI catalytic domain, that need to dimerize to form an active entity able to cleave a DNA target sequence.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Influence of DNA Methylation on the Binding Affinity and Nuclease Activity of I-CreI Towards its DNA Target

The effect of DNA methylation on the binding affinity and nuclease activity of I-CreI (SEQ ID NO: 1) towards its DNA target C1221 (SEQ ID NO: 5) was investigated. In vitro binding and cleavage assays using I-CreI (SEQ ID NO: 1) and its palindromic DNA target C1221 (SEQ ID NO: 5) containing either 0 or 4 methylated CG on both strands were performed.

Material and Methods

Cloning, overexpression and purification of Cterm His-tag I-CreI The coding sequence of I-CreI (SEQ ID NO: 1) was subcloned into the kanamycin resistant pET-24 vector MCS located upstream a 6×His-tag coding sequence. Recombinant plasmid containing the coding sequence of Cterm His-tag I-CreI was then transformed into E. coli BL21 (Invitrogen) and positive transformants were selected on LB-agar medium supplemented by kanamycin.

To overexpress the Cterm His-tag I-CreI, 800 mL of E. coli BL21 cultures were grown in the presence of kanamycin to mid-exponential phase and were then induced by adding IPTG (Sigma) to a final concentration of 750 μM. After induction, cell growth proceeded for 14 hours at 20° C. Cells were then harvested by centrifugation at 4000 rpm for 30 min and suspended in 25 ml of lysis buffer (20 mM Tris-HCl, 500 mM NaCl, 10 mM imidazol, pH8). Extraction of soluble proteins was performed by 8 series of 30 seconds sonication pulses in the presence of Complete EDTA-free antiprotease at 4° C. The resulting cell extract were clarified by centrifugation at 13000 rpm for 30 min at 4° C. and supernatant was used as crude extract for purification. Crude extract were loaded onto a 1 mL Bio-Scale IMAC cartridge (Bio-Rad) equilibrated with lysis buffer using the profinia system (Bio-Rad). The column was then washed with 3 column volumes of lysis buffer followed by 3 column volumes of the same buffer plus 40 mM imidazol and 1M NaCl. This second washing step efficiently removed the majority of protein contaminants and non-specific DNA bound to I-CreI. I-CreI was eluted with 250 mM imidazol and directly desalted on a 10 mL Bio-Scale P-6 desalting column (Bio-Rad) equilibrated with desalting buffer (20 mM Tris-HCl, 100 mM NaCl, 1 mM EDTA, pH8). Fraction containing I-CreI (90% homogeneity, ˜1 mg/mL) were aliquoted, flash frozen in liquid nitrogen and stored at −80° C.

In Vitro Binding Assay

Fluorescein labeled C1221 oligonucleotides were synthesized and HPLC-purified by Eurogentec. To prepare C1221 duplex, C1221 forward labeled with Fluorescein on its 5′ end (5′Fluo_C1221_Forward, SEQ ID NO:4) was mixed with 1 equivalent of C1221 Reverse (SEQ ID NO: 5) in 100 mM Tris-HCl, 50 mM EDTA, 150 mM NaCl, pH8. The mixture was heated to 95° C. for 2 min and then cooled down to 25° C. over 1 hour. C1221 duplex and fluorescein final concentrations were assessed by spectrophotometry using their respective extinction coefficients ε₂₆₀ₙₘ=62900 M⁻¹ cm⁻¹ and ε₄₉₅ₙₘ=83000 M⁻¹ cm⁻¹. As expected, we obtained a ratio [C 1221 duplex]/[fluorescein]˜1. The same procedure was used to prepare the fully methylated C1221 duplex from 5′Fluo_C1221_{—}4Me Forward and C1221_{—}4Me_Reverse (SEQ ID NOs: 12 and 13 respectively).

To investigate the binding of I-CreI to C1221 duplex, 50 nM of C1221 duplex was incubated with increasing concentrations of I-CreI (from 0 to 1.5 μM) in binding buffer (10 mM Tris-HCl, 150 mM NaCl, 10 mM CaCl₂, 10 mM DTT, pH8) at 25° C. After 30 minutes incubation, the fluorescence anisotropy of the mixture was recorded with a Pherastar Plus (BMG Labtech) operating in fluorescence polarization end point mode with excitation and emission wavelengths set to 495 and 520 nm respectively. Apparent dissociation constant were estimated by determining the [I-CreI]₅₀. [I-CreI]₅₀ is defined as the concentration of 1-CreI needed to reach 50% of the final fluorescence anisotropy.

In Vitro Cleavage Assay

To investigate the influence of C1221 methylation on the nuclease activity of I-CreI, an in vitro cleavage assay with either unmethylated or fully methylated C1221 duplex (SEQ ID NOs: 4-5 and 12-13 respectively) was performed. A constant amount of C1221 duplex (50 nM) was incubated with increasing concentration of I-CreI (0 to 2 μM) in a total volume of 25 μL of reaction buffer (10 mM Tris-HCl, 150 mM NaCl, 10 mM MgCl₂, pH8). Cleavage reaction was allowed to proceed 1 hour at 37° C. and then stopped by addition of 5 μL of 6× stop buffer (45% glycerol, 95 mM EDTA, 1.5% (w/v) SDS, 1.5 mg/mL proteinase K and 0.048% (w/v) bromophenol blue) followed by an hour incubation at 37° C. Cleaved and uncleaved DNA products were separated by PAGE using a TGX Any kD precast gel (Bio-Rad), stained with SYBR Green and then quantified using Quantity One software (Bio-Rad).

Results

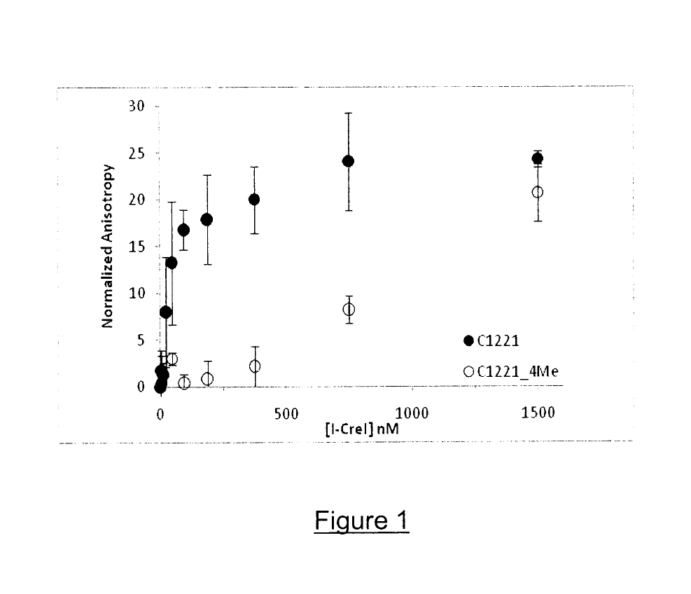

To investigate the influence of DNA methylation on the binding affinity of I-CreI for its specific DNA target C1221, the apparent dissociation constant values ([I-CreI]_{0.5}) for unmethylated and fully methylated C1221 with I-CreI were determined in vitro. To do so, fluorescence anisotropy of fluorescein-labeled C1221 duplex was recorded in the presence of increasing amounts of I-CreI (FIG. 1). In the case of unmethylated C1221, we observed an increase of fluorescence anisotropy that leveled up at saturating concentration of I-CreI. This pattern is consistent with a binding equilibrium between I-CreI and 0221. The apparent dissociation constant of this binding equilibrium can be estimated by determining the concentration of I-CreI needed to reach 50% of the final fluorescence anisotropy. This value, named [I-CreI]₅₀, was estimated to 30 nM.

In the case of fully methylated C1221, fluorescence anisotropy remained almost constant at low concentration of I-CreI ([I-CreI]<500 nM) and increased steadily at higher concentrations. In addition, binding of I-CreI to methylated C1221 was not completed under our experimental conditions. Indeed, any signal saturation in the presence of a large excess of I-CreI with respect to C1221 was observed. Nevertheless, the [I-CreI]₅₀ value for fully methylated C1221 was estimated to be roughly 1000 nM. These results indicate that the affinity of I-CreI for fully methylated C1221 is significantly lower than for unmethylated C1221.

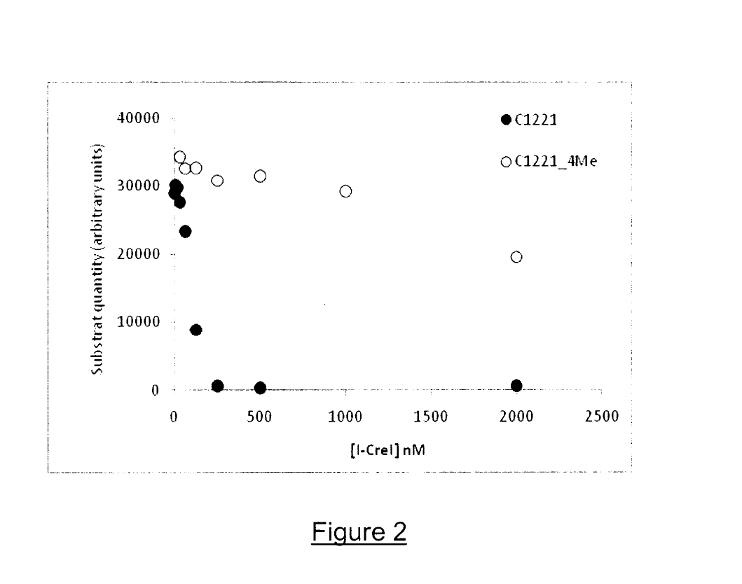

To test the influence of C1221 methylation on I-CreI nuclease activity, an in vitro cleavage assay with either unmethylated or fully methylated C1221 (FIG. 2) as substrates was performed. In the case of unmethylated C1221, the results show that the amount of C1221 substrate decreases almost linearly with respect to I-CreI concentration until being totally cleaved in the presence of 250 nM of I-CreI (4 eq of I-CreI with respect to C1221). The C₅₀ (concentration of I-CreI needed to cleave 50% of C1221) is estimated to 100 nM. This indicates that I-CreI efficiently cleaves C1221. On the other hand, the fully methylated C1221 was cleaved much less efficiently than C1221 with a C₅₀>2 μM. In addition, cleavage reaction didn't go to completion under these experimental conditions. Therefore, taken together, the results indicate that C1221 methylation significantly inhibits the nuclease activity of I-CreI.

### Example 2

### Influence of DNA Methylation on the Nuclease Activity of I-CreI D75N Towards its DNA Target C1221

The effect of DNA methylation on the nuclease activity of I-CreI D75N (SEQ ID NO: 22) towards its DNA target C1221 (SEQ ID NO: 5) was investigated. In vitro cleavage assay using recombinant I-CreI D75N and its palindromic target C1221 containing either 0, 1, 2 or 3 methylated CG on both strands was performed.

Material and Methods

Cloning, Overexpression and Purification of I-CreI D75N

To clone, overexpress and purify I-CreI D75N, the same procedure as in example 1 was used.

In Vitro Cleavage Assay

To investigate the influence of C1221 methylation on the nuclease activity of I-CreI D75N, in vitro cleavage assay with C1221 duplex containing either 0, 1, 2 or 3 methylated CG (SEQ ID NOs: 4-5, 6-7, 8-9, 10-11 respectively) was performed, according to the procedure described for I-CreI wild type.

Results

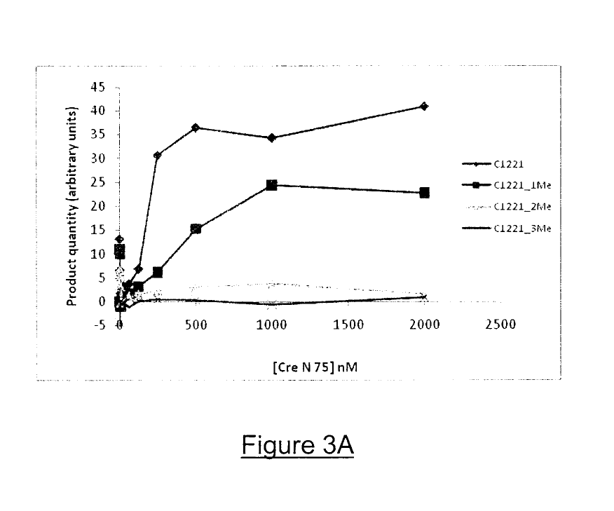

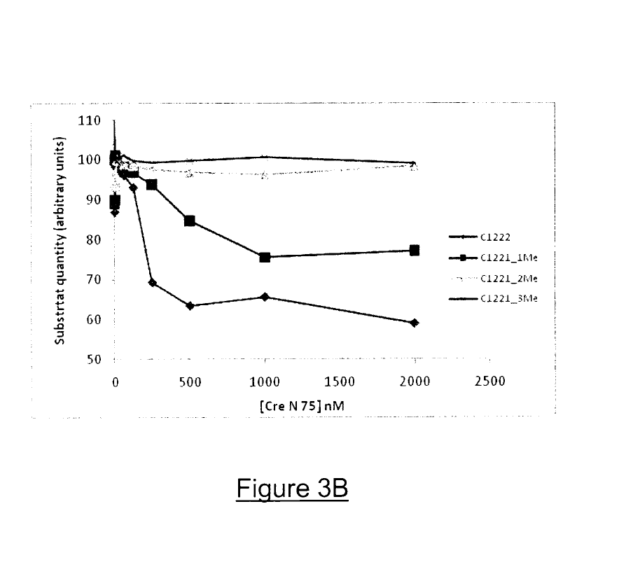

To test the influence of C1221 methylation on I-CreI D75N nuclease activity, in vitro cleavage assay with C1221 containing either 0, 1, 2, 3 methylated CG as substrates was performed. In the case of unmethylated C1221, the amount of C1221 cleaved product increased almost linearly with respect to I-CreI D75N concentration and leveled up in the presence of about 500 nM of I-CreI (FIG. 3 top panel). Accordingly, an anticorrelation between product and substrate variations as a function of I-CreI D75N (FIG. 3 top and bottom panels) was observed. The C₅₀ (concentration of I-CreI D75N needed to cleave 50% of C1221) was estimated to be 100-150 nM. Accordingly, this C₅₀ value is similar to the one obtained in example 1 in the same experimental conditions. Interestingly, increasing methylation of C1221 gradually increased the C₅₀. Indeed addition of one methyl group on both strand increased C₅₀ by about 3 folds while addition of two and three methyl groups resulted in a more than 10 folds increase of C₅₀. The nuclease activity of I-CreI D75N is then strongly affected by C1221 methylation.

### Example 3

### Influence of DNA Methylation on the Binding Affinity and Nuclease Activity of I-Cre I Wild Type for its Specific DNA Target C1234

In this example, the effect of DNA methylation on the binding affinity and nuclease activity of I-Cre I wild type (SEQ ID NO: 1) for its specific target was investigated. To do so in vitro binding and cleavage assays were performed using recombinant I-Cre I wild type and its natural target C1234 (forward C1234, SEQ ID NO: 31) containing different amounts of methylated CGs.

Material and Methods

Cloning, Overexpression and Purification of Cterm His-Tag I-Cre I Wild Type

The coding sequence for I-Cre I wild type (SEQ ID NO: 1) was subcloned into the kanamycin resistant pET-24 vector MCS located upstream a 6×His-tag coding sequence. Recombinant plasmid containing the coding sequence of Cterm His-tag I-Cre I wild type was then transformed into E. coli BL21 (Invitrogen) and positive transformants were selected on LB-agar medium supplemented by kanamycin.

To overexpress Cterm His-tag I-Cre I wild type (named I-Cre I in the following), 800 mL of E. coli BL21 cultures were grown in the presence of kanamycin to mid-exponential phase and were then induced by adding IPTG (Sigma) to a final concentration of 750 μM. After induction, cell growth proceeded for 14 hours at 20° C. Cells were then harvested by centrifugation at 4000 rpm for 30 min and suspended in 25 ml of lysis buffer (20 mM Tris-HCl, 500 mM NaCl, 10 mM imidazol, pH8). Extraction of soluble proteins was performed by 8 series of 30 seconds sonication pulses in the presence of Complete EDTA-free antiprotease at 4° C. The resulting cell extracts were clarified by centrifugation at 13000 rpm for 30 min at 4° C. and supernatants were used as crude extracts for purification. Crude extracts were loaded onto a 1 mL Bio-Scale IMAC cartridge (Bio-Rad) equilibrated with lysis buffer using the profinia system (Bio-Rad). The column was then washed with 3 column volumes of lysis buffer followed by 3 column volumes of the same buffer plus 40 mM imidazol and 1M NaCl. This second washing step efficiently removed the majority of protein contaminants and non-specific DNA bound to I-Cre I. I-Cre I was eluted with 250 mM imidazol and directly desalted on a 10 mL Bio-Scale P-6 desalting column (Bio-Rad) equilibrated with desalting buffer (20 mM Tris-HCl, 100 mM NaCl, 1 mM EDTA, pH8). Fractions containing I-Cre I (90% homogeneity, ˜1 mg/mL) were aliquoted, flash frozen in liquid nitrogen and stored at −80° C.

In Vitro Binding Assay

Fluorescein labeled C1234 oligonucleotides were synthesized and HPLC-purified by Eurogentec. To prepare C 1234 duplex corresponding to I-Cre I double strand DNA wild type target, C1234 forward (SEQ ID NO: 31, “a” strand below) labeled with Fluorescein on its 5′ end was mixed with 1 equivalent of C1234_reverse (SEQ ID NO: 32, “b” strand below) in 100 mM Tris-HCl, 50 mM EDTA, 150 mM NaCl, pH8. The mixture was heated to 95° C. for 2 min and then cooled down to 25° C. over 1 hour. C1234 duplex was eventually purified by anion exchange chromatography using a miniQ PE column (GE healthcare) pre-equilibrated with buffer A (20 mM Tris-HCl, pH7.4). Single stranded oligonucleotides and other contaminants were first discarded using a 0 to 360 mM NaCl step gradient and elution of pure C1234 duplex was performed with a 360-1000 mM NaCl linear gradient (5 column volumes). C 1234 duplex and fluorescein final concentrations were assessed by spectrophotometry using their respective extinction coefficients ε₂₆₀ₙₘ=62900 M⁻¹ cm⁻¹ and ε₄₉₅ₙₘ=83000 M⁻¹ cm⁻¹. As expected, a ratio [C1234 duplex]/[fluorescein]˜1 was obtained. The same procedure was used to prepare the different methylated forms of C 1234 duplex (C 1234_Me full composed of SEQ ID NO: 35+SEQ ID NO: 38, C1234_Me-6a/-5b composed of SEQ ID NO: 33+SEQ ID NO: 36, C1234_Me-3a/-2b composed of SEQ ID NO: 34+SEQ ID NO: 37, C1234_Me-3a composed of SEQ ID NO: 34+SEQ ID NO: 32, C1234_Me-2b composed of SEQ ID NO: 31+SEQ ID NO: 37, respectively where “a” and “b” designate each of the DNA strands; for example C1234 Me-6a/-5b is a duplex with methylations at position −6 on strand “a” and at position −5 on strand “b”, respectively) and the random target corresponding to a stretch of 12 GA repeats, (forward, SEQ ID NO: 39 and reverse, SEQ ID NO: 40). To investigate the binding of I-Cre I to C1234 duplex, 25 nM of C1234 duplex was incubated with increasing concentrations of I-Cre I (from 0 to 400 nM) in binding buffer (10 mM Tris-HCl, 400 mM NaCl, 10 mM CaCl₂, 10 mM DTT, pH8) at 25° C. After 30 minutes incubation, the fluorescence anisotropy of the mixture was recorded with a Pherastar Plus (BMG Labtech) operating in fluorescence polarization end point mode with excitation and emission wavelengths set to 495 and 520 nm respectively. Apparent dissociation constants were determined by fitting raw data by an hyperbolic function (Aₓ=(A_{∞}*[Meganuclease])/(K_{d}+[Meganuclease]) with Aₓ, the fluorescence anisotropy value obtained at a given meganuclease concentration, A_{∞}, the fluorescence anisotropy value obtained at saturating concentration of meganuclease and K_{d} the dissociation constant of the equilibrium studied).

In Vitro Cleavage Assay

To investigate the influence of C1234 methylation on the nuclease activity of I-Cre I, in vitro single turn over cleavage assays were performed with either unmethylated or methylated C1234 duplexes (unmethylated C1234 composed of SEQ ID NO: 31+SEQ ID NO: 32, C1234_Me full composed of SEQ ID NO: 35+SEQ ID NO: 38, C1234_Me-6a/-5b composed of SEQ ID NO: 33+SEQ ID NO: 36, C1234_Me-3a/-2b composed of SEQ ID NO: 34+SEQ ID NO: 37, C1234_Me-3a composed of SEQ ID NO: 34+SEQ ID NO: 32, C1234_Me-2b composed of SEQ ID NO: 31+SEQ ID NO: 37, respectively). A constant amount of C1234 duplex (50 nM) was incubated with an excess of I-Cre I (1.5 μM, final concentration) in the reaction buffer (10 mM Tris-HCl, 150 mM NaCl, pH8) at 37° C. Cleavage reaction was triggered by the addition of MgCl₂ and then stopped after different time lengths by the addition of the stop buffer (45% glycerol, 95 mM EDTA, 1.5% (w/v) SDS, 1.5 mg/mL proteinase K and 0.048% (w/v) bromophenol blue, final concentrations). This was followed by one hour incubation at 37° C. to digest I-Cre I and release free DNA molecules. Cleaved and uncleaved DNA products were separated by PAGE using a TGX Any kD precast gel (Bio-Rad), stained with SYBR Green and then quantified using Quantity One software (Bio-Rad).

Results

In Vitro Binding Assay

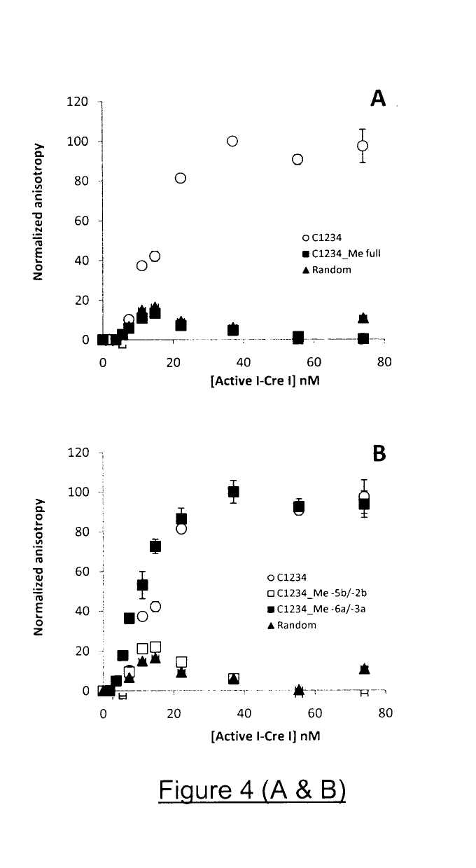

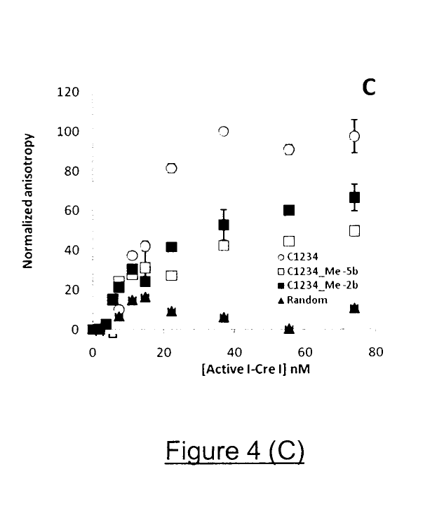

To investigate the influence of DNA methylation on the binding affinity of I-Cre I for its natural DNA target C1234, the dissociation constant values (IQ) for unmethylated and methylated C1234 with I-Cre I were determined in vitro. To do so, fluorescence anisotropy of fluorescein-labeled C1234 duplex was recorded in the presence of increasing amounts of I-Cre I (FIG. 4A, open circles). In the case of unmethylated C1234, an increase of fluorescence anisotropy was observed that leveled up at saturating concentration of I-Cre I. This pattern was consistent with a tight binding equilibrium between I-Cre I and C1234. The dissociation constant of this binding equilibrium can be estimated to be ≦2.5 nM.

In the case of fully methylated C1234, fluorescence anisotropy remained almost constant in the presence of up to 400 nM of I-Cre I (FIG. 4A, closed squares, data shown up to 80 nM). Binding of I-Cre I to methylated C1234 could be not completed under our experimental conditions as we didn't observe any signal saturation in the presence of a large excess of I-Cre I with respect to C1234. Nevertheless, the K_{d} value for fully methylated C1234 could be estimated >1000 nM. These results indicated that the affinity of I-Cre I for fully methylated C1234 was at least 400 times lower than for unmethylated C1234. Therefore, CGs methylation of C1234 strongly affects its affinity for I-Cre I.

To decipher the inhibitory effect of CGs methylation on I-Cre I binding capacity, a stepwise approach was undertaken, first asking whether this inhibitory effect was strand dependent and, secondly, position dependent.

To investigate the strand dependence of this inhibitory effect, affinity of I-Cre I for hemimethylated C1234 was first compared (either on “a” or “b” strands methylated in positions −6a/−3a and composed of SEQ ID NO: 35+SEQ ID NO: 32 or in positions −5b/−2b and composed of SEQ ID NO: 31+SEQ ID NO: 38, respectively, see FIG. 4B). Fluorescence anisotropy results showed that C1234_Me-6a/-3a (composed of SEQ ID NO: 35+SEQ ID NO: 32) displayed similar affinity for I-Cre I than unmethylated C1234 (composed of SEQ ID NO: 31+SEQ ID NO: 32, FIG. 4B, table I). Interestingly, it was found that C1234 Me-5b/-2b (composed of SEQ ID NO: 31+SEQ ID NO: 38) had a much lower affinity for I-Cre I than unmethylated C1234 (composed of SEQ ID NO: 31+SEQ ID NO: 32) and C1234_Me-6a/-3a (composed of SEQ ID NO: 35+SEQ ID NO: 32; FIG. 4B, table I). Indeed, no signal saturation was observed in the presence of a large excess of I-Cre I.

This indicated that methylation of C1234 “b” strand significantly affected its affinity for I-Cre I whereas a methylation effect associated to C 1234 “a” strand was not detectable.

To further our understanding on the position dependent inhibitory effect of CGs methylation, the affinity of I-Cre I for C1234 methylated either in position −5b, either in position −2b (composed of SEQ ID NO: 31+SEQ ID NO: 36 or composed of SEQ ID NO: 31+SEQ ID NO: 37, FIG. 4C) was compared. Results showed that methylation of both positions affected I-Cre I affinity for C1234 by a factor ≦10 with respect to unmethylated C1234 (composed of SEQ ID NO: 31+SEQ ID NO: 32, table I). Thus, taken together, these results indicated that I-Cre I binding capacity is impaired by methylation of C 1234 “b” strand.

In Vitro Cleavage Assay

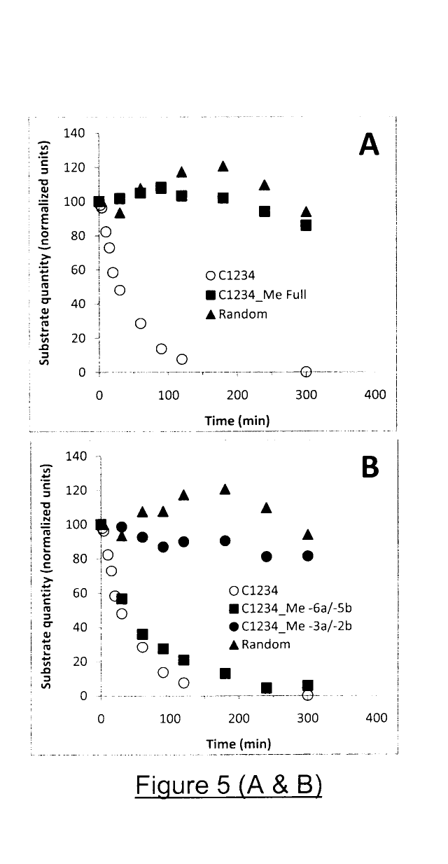

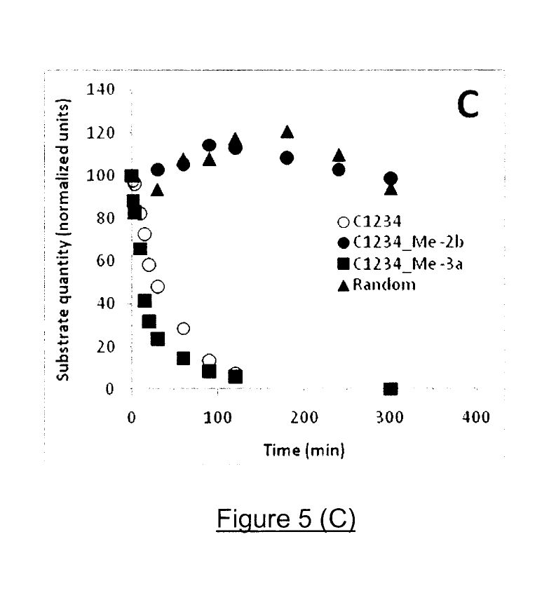

To test the influence of C 1234 methylation on I-Cre I cleavage activity, single turn over cleavage assays in vitro were performed with unmethylated (composed of SEQ ID NO: 31+SEQ ID NO: 32) or with different methylated forms of C1234 (FIG. 5). In these assays C1234 was premixed to a large excess of recombinant meganucleases before addition of MgCl₂. After different time lengths, the reaction was quenched by addition of a stop buffer, the remaining substrate was separated from the reaction product and then quantified. In these conditions, substrate disappearance was a first order process. The rate constant of this process corresponded to the turn over number (k_{cat}) of the meganuclease. Turn over number measurement was not affected by affinity differences between methylated and unmethylated C1234 for I-Cre I because in our experimental conditions, the totality of C1234 was bound to the meganuclease at the beginning of reaction. In addition, this measurement was not affected by the rate limiting step of product release (Wang J, Kim H H, Yuan X, Herrin D L: Purification, biochemical characterization and protein-DNA interactions of the I-CreI endonuclease produced in Escherichia coli. Nucleic Acids Res 1997, 25:3767-3776) because the complex I-Cre I:cleaved C1234 product was artificially disrupted by the proteinase K and SDS present in the stop buffer.

In the case of unmethylated C1234, results showed that the disappearance of C1234 substrate followed a monoexponential behavior that was characteristic of a first order process. The rate constant of this process was determined to be k=0.025 min⁻¹ (FIG. 5A, table I). This indicated that I-Cre I efficiently cleaved C1234 with a k_{cat}=0.025 min⁻¹ as reported earlier by Wang & al (Wang J, Kim H H, Yuan X, Herrin D L: Purification, biochemical characterization and protein-DNA interactions of the I-CreI endonuclease produced in Escherichia coli. Nucleic Acids Res 1997, 25:3767-3776). On the other hand, when fully methylated C1234 (composed of SEQ ID NO: 35+SEQ ID NO: 38) was assayed, a much slower process was observed with k_{cat} estimated to be <0.0001 min⁻¹, indicating that C 1234 methylation significantly inhibited the nuclease activity of I-Cre I (FIG. 5A, table I).

To investigate in more details the inhibitory effect of methylation toward I-Cre I cleavage activity, once again a stepwise approach was used. The effect of methylated CGs located outside the cleavage region (C1234 methylated in positions −6a/−5b, composed of SEQ ID NO: 33+SEQ ID NO: 36) was first compared to those located within the cleavage region (C1234 methylated in positions −3a and −2b, composed of SEQ ID NO: 34+SEQ ID NO: 37). Results showed that methylation of CGs located outside the cleavage region did not affect I-Cre I catalytic activity as no significant k_{cat} difference could be detected when compared to the k_{cat} obtained with unmethylated C1234 (FIG. 5B, filled squares). On an other hand, these data showed that methylation of CGs located within the cleavage region, strongly affected the catalytic activity of I-Cre I (FIG. 5B, filled circles). To further understand this inhibitory effect, the effect of different single CG methylation was investigated (C1234 either methylated in position −3a (composed of SEQ ID NO: 34+SEQ ID NO: 32) or methylated in position −2b (composed of SEQ ID NO: 31+SEQ ID NO: 37) on I-Cre I cleavage activity. Results showed that methylation of −3a position didn't affect 1-Cre I catalytic activity whereas methylation of −2b almost totally inhibited it (filled squares and filled circles respectively.

TABLE I
K_{d} and k_{cat} of I-Crel for different C1234 DNA targets.

| | Meganuclease | | | | | |
|---|---|---|---|---|---|---|
| | I-Cre I | | | | | |
| DNA target | C1234 | C1234_Me full | C1234_Me-5b/-2b | C1234_Me-6a/-3a | C1234_Me-6a/-5b | C1234_Me-3a/-2b |
| K_{d} (nM) | ≦2.5 | >1000 | >1000 | ≦2.5 | — | — |
| k_{cat} (min−1) | 0.025 ± 0.002 | <0.0001 | — | — | 0.02 ± 0.007 | 0.0002 ± 0.003 |

| | Meganuclease | | | | |
|---|---|---|---|---|---|
| | I-Cre I | | | | |
| DNA target | C1234_Me-3a/-2b | C1234_Me-3a | C1234_Me-2b | C1234_Me-5b | Random |
| K_{d} (nM) | — | — | 40.8 | 24.0 | >1000 |
| k_{cat} (min−1) | 0.0002 ± 0.003 | 0.057 ± 0.006 | <0.0001 | — | <0.0001 |

Model of I-Cre I:C1234_Me Full

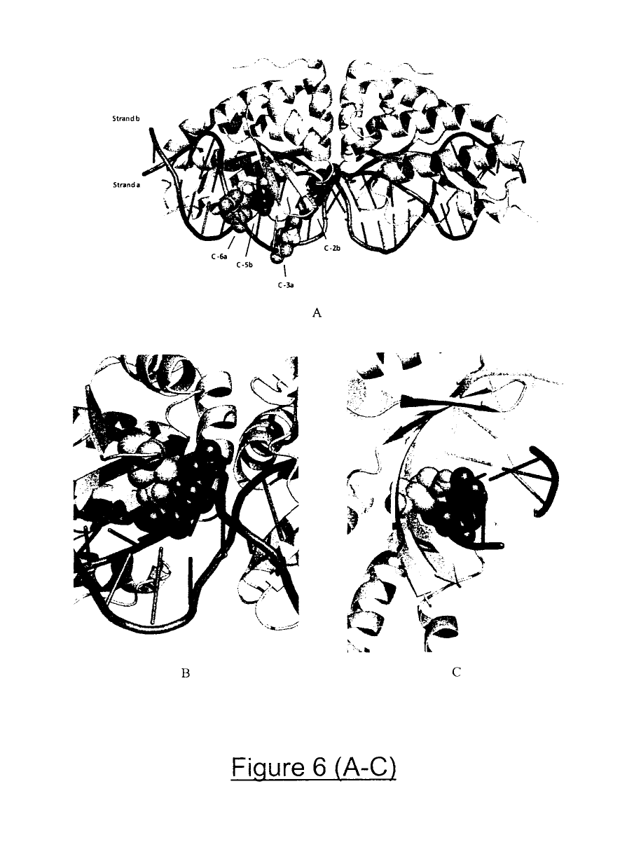

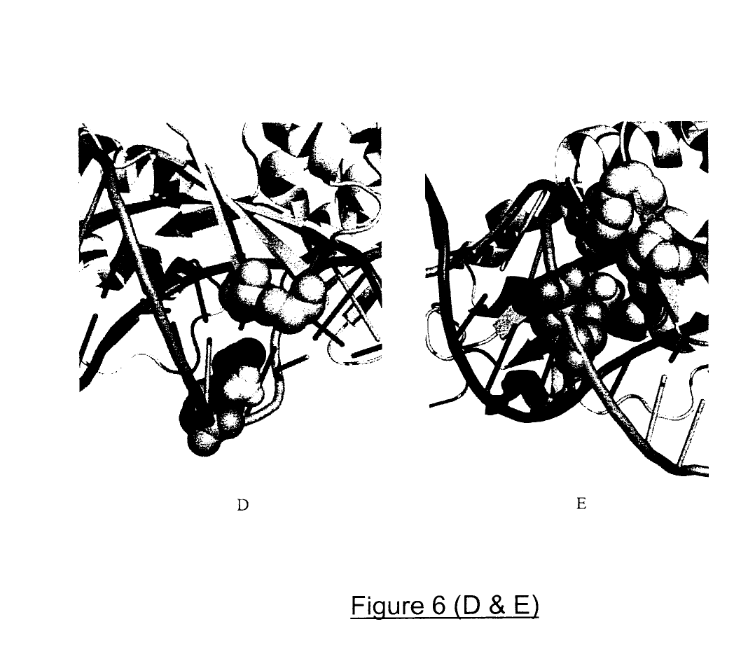

To understand the inhibition of I-Cre I cleavage activity by CG methylation at a molecular level, the 3D model of I-Cre I bound to the fully methylated C1234 (composed of SEQ ID NO: 35+SEQ ID NO: 38) was investigated. The complex I-Cre I:C1234_Me full was modeled using Pymol and I-Cre I:C1234 structure (PDB ID, 1G9Y) as a template (FIG. 6). For the sake of clarity, “a” and “b” strands were colored in cyan and purple respectively, methylated cytosines were displayed in VDW spheres and their 5′ methyl moieties were highlighted either in pale cyan (C-6a and C-3a) or in pale purple (C-5b and C-2b). From this model, it was found that methyl moieties of cytosines-5b and -2b were in steric clash with Ile 24 and Valine 73 side chains respectively (FIGS. 6B and 6C). These steric clashes are very likely to impair the interaction between I-Cre I and C 1234 and thus to decrease the affinity for one another. Interestingly, these steric clashes were not observed with the methylated cytosines-6a and -3a. Indeed, the methyl moiety of cytosine-3a pointed toward the solvant and was free of any interaction with I-Cre I backbone, even with the closest amino acids Arg 70 and Gly 71. Similarly, the methyl moiety of cytosine-6a didn't display any obvious clash with the closest amino acids Tyr 66 and Arg 68. Therefore, in contrast to cytosines-5b and -2b, the presence of methyl moieties on cytosines-3a and -6a is unlikely to impair the interaction between I-Cre I and C 1234. These observations are consistent with biochemical data which clearly showed the negative impact of C 1234 b strand methylation on binding capacity and cleavage activity of I-Cre I.

### Example 4

### Influence of DNA Methylation In Vitro and In Vivo on XPC4 Meganuclease

a) Influence of DNA Methylation on the Binding Affinity and Nuclease Activity In Vitro of XPC4 Towards its Specific DNA Target XPC4.1

To investigate the effect of DNA methylation on the binding affinity and cleavage activity of a meganuclease towards its DNA target, an engineered meganuclease named XPC4 specifically designed to cleave xeroderma pigmentosum group C gene (XPC) was used. In this example, in vitro binding and cleavage assays were performed using recombinant XPC4 (SEQ ID NO: 2) and its natural target XPC4.1 containing either 0 methylated CG (composed of SEQ ID NO: 14+SEQ ID NO: 15) or 4 methylated CGs at positions −11a, −10b and +10a, +11b respectively (composed of SEQ ID NO: 16+SEQ ID NO: 17).

Material and Methods

Cloning, Overexpression and Purification of XPC4

Cloning, overexpression and purification of XPC4 (SEQ ID NO: 2) were performed according to the procedure described in example 3.

Binding Assay

To determine the affinity of XPC4 for its unmethylated and methylated specific target XPC4.1, binding assays were performed according to the procedure described in example 3 using 5′ end fluorescein-labeled unmethylated and fully methylated XPC4.1 oligonucleotides (composed of SEQ ID NO: 14+SEQ ID NO: 15 and SEQ ID NO: 16+SEQ ID NO: 17, respectively).

In Vitro Cleavage Assay

To investigate the influence of XPC4.1 methylation on the nuclease activity of XPC4, in vitro single turn over cleavage assays were performed with either unmethylated (composed of SEQ ID NO: 14+SEQ ID NO: 15) or methylated XPC4.1 (composed of SEQ ID NO: 16+SEQ ID NO: 17) according to the procedure described in example 3.

Results

In Vitro Binding Assay

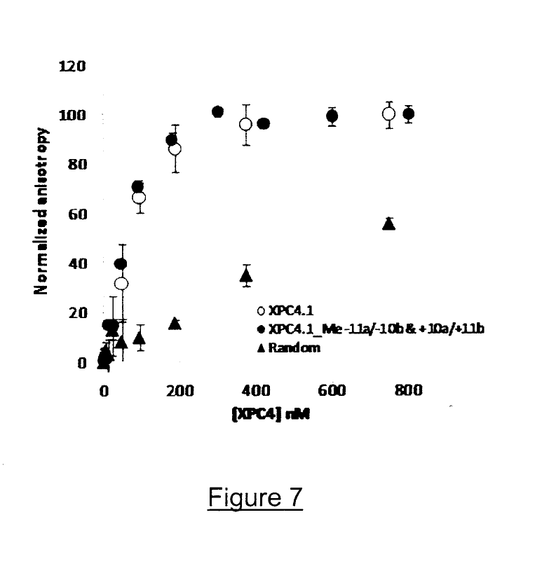

To investigate the influence of DNA methylation on the binding affinity of XPC4 for its specific DNA target (XPC4.1), the dissociation constant values (K_{d}) for methylated and unmethylated XPC4.1 with XPC4 were determined in vitro. Fluorescence anisotropy of fluorescein-labeled XPC4.1 duplex was recorded in the presence of increasing amounts XPC4 (FIG. 7). In the case of unmethylated XPC4.1 (composed of SEQ ID NO: 14+SEQ ID NO: 15), fluorescence anisotropy increased and then leveled up at saturating concentration of XPC4. This pattern was consistent with a binding equilibrium between XPC4 and XPC4.1. The dissociation constant of this binding equilibrium could be estimated to 108±21 nM (table II).

Regarding the fully methylated XPC4.1 (composed of SEQ ID NO: 16+SEQ ID NO: 17), fluorescence anisotropy varies according to the same pattern and the dissociation constant of this binding equilibrium could be estimated to 127±22 nM (table II). These results indicated that in these experimental conditions the affinity of XPC4 for fully methylated XPC4.1 was slightly lower, although not significant, than for unmethylated XPC4.1.

In Vitro Cleavage Assay

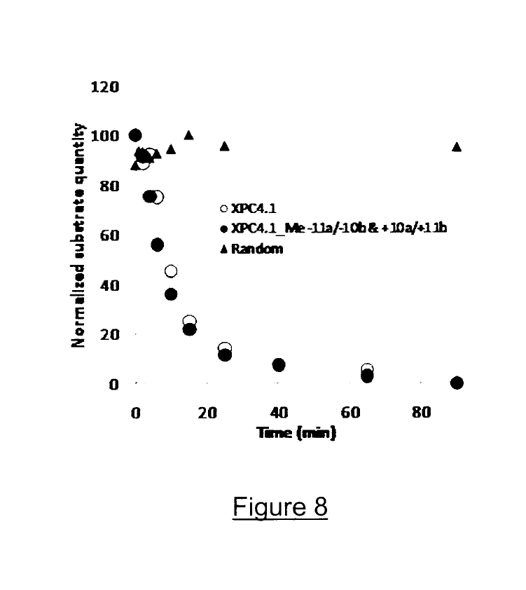

To test the influence of XPC4.1 methylation on XPC4 cleavage activity, single turn over cleavage assays were performed in vitro with unmethylated (composed of SEQ ID NO: 14+SEQ ID NO: 15) or with fully methylated (composed of SEQ ID NO: 16+SEQ ID NO: 17) forms of XPC4.1 as substrates as described in example 3. In the case of unmethylated XPC4.1, results showed that the disappearance of XPC4.1 substrate followed a monoexponential behavior that was characteristic of a first order process (FIG. 8). The rate constant of this process was determined to be k=0.078±0.01 min⁻¹ (table II). This indicated that XPC4 efficiently cleaved XPC4.1 with a k_{cat}=0.078±0.01 min⁻¹. Interestingly, when fully methylated XPC4.1 was assayed, a similar trend with k_{cat} estimated to be 0.1±0.01 min⁻¹ was observed, indicating that XPC4.1 methylation didn't significantly inhibit the nuclease activity of XPC4 (FIG. 8, table II) in our experimental conditions.

Taken together, these results showed that methylation of XPC4.1 at positions −11a, −10b, +10a and +11b, didn't affect the cleavage activity of XPC4 in vitro.

TABLE II
K_{d} and k_{cat} of XPC4 meganuclease for XPC4.1 DNA targets.

| | XPC4 | | |
|---|---|---|---|
| | | XPC4.1_Me | |
| Meganuclease | | −11a/−10b & | |
| DNA target | XPC4.1 | +10a/+11b | Random |
| K_{d} (nM) | 108 ± 21 | 127 ± 22 | >1000 |
| k_{cat} (min−1) | 0.078 ± 0.01 | 0.1 ± 0.01 | <0.0001 |

b) Effect of 5-Aza-Deoxycytidine on the Cleavage Efficiency of the XPC4 Meganuclease In Vivo

To investigate the effect of DNA methylation on meganuclease cleavage, an engineered meganuclease called XPC4 (SEQ ID NO: 2) designed to cleave a DNA sequence 5′-TCGAGATGTCACACAGAGGTACGA-3′ (SEQ ID NO: 24) present in the Xeroderma Pigmentosum group C gene (XPC) was used. The XPC4 target is found in a relatively CpG rich environment (with 23 CpG in 1 kb of surrounding sequence), and contains two CpG motives. These CpG motives are potentially methylated in cells. The impact of a methylase inhibitor on the methylation profile of these two CpG motives was measured, as well as on the cleavage efficiency of XPC4 target by the XPC4 meganuclease.

Materials and Methods

Cell Transfection

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS) supplemented with 5-aza-deoxycytidine. The next day, cells were transfected with 3 μg of meganuclease expression vector in the presence of 5-aza-deoxycytidine and Lipofectamine 2000 transfection reagent (Invitrogen) according to the manufacturer's protocol.

### 5-aza-2-deoxycytidine Treatment

Cells were pre-treated with 5-aza-deoxycytidine at 0.2 μM or 1 μM 48 hours before transfection and the treatment was maintained 48 hours post-transfection. As a control, cells not treated with 5-aza-2-deoxycytidine (NT) were used. Extraction of genomic DNA was performed 48 hours after transfection.

Monitoring of DNA Methylation by Bisulfite Treatment and DNA Sequencing

To assess the level DNA methylation, DNA sequencing was performed after a bisulfite treatment according to the instructions of the manufacturer (EZ DNA methylation-Gold Kit, Zymo Research). After genomic DNA extraction, the XPC4 target locus was amplified by PCR with specific primers

F1:
(SEQ ID NO: 25)
5′-GTTGGTATAGATTAGTGGTTAGAGGTGTTTTG-3′
and
R1:
(SEQ ID NO: 26)
5′-CTTAAAACCCCTAACAACCAAAACCTTACC-3′.

The PCR product was sequenced directly with primers:

F2:
(SEQ ID NO: 27)
5′-GTGGGTATGTGTAGATTGTGTGTAYGGTGTG-3′
and
R2:
(SEQ ID NO: 28)
5′-CTCCAAATCTTCTTTCTTCTCCCTATCC-3′.

Monitoring of Meganuclease-Induced Mutagenesis by Deep Sequencing

After genomic DNA extraction, the XPC4 target locus was amplified with specific primers flanked by specific adaptator needed for HTS sequencing on the 454 sequencing system (454 Life Sciences)

F3:
(SEQ ID NO: 29)
5′-CCATCTCATCCCTGCGTGTCTCCGACTCAGTGCCAAGAGGCAAGAA
AATGTGCAGC-3′
and
R3:
(SEQ ID NO: 30)
5′-BiotineTEG/CCTATCCCCTGTGTGCCTTGGCAGTCTCAGGCTGG
GCATATATAAGGTGCTCAA-3′.

5000 to 10 000 sequences per sample were analyzed.

Results

293H cells were transfected with XPC4 meganuclease or empty vector in presence or absence of 5-aza-2′deoxycytidine, at the concentration of 0.2 or 1 μM.

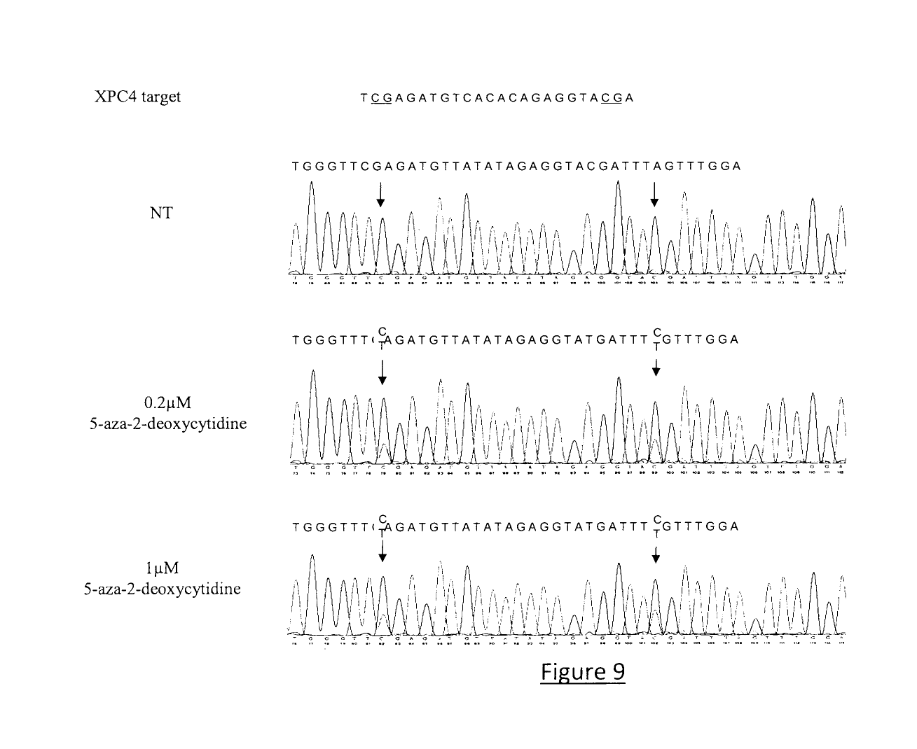

In order to determine the methylation status of the two CpG motives present in the XPC4 target sequence, genomic DNA was extracted, and treated with bisulfite. Bisulfite treatment is based on a chemical reaction of sodium bisulfite with DNA that converts unmethylated cytosines into uracil whereas methylated cytosines remain unchanged. DNA was then amplified by PCR and sequenced. Examples of sequences are shown in FIG. 9. In absence of 5-aza-2′deoxycytidine treatment, no cytosine conversion was observed in XPC4 target sequence, showing that both CpG were methylated in the vast majority of the cells. After 5-aza-2′deoxycytidine treatment, we observed dual peaks in the chromatogram (FIG. 9), showing that in the treated cell population, the two CpG could be methylated or unmethylated. For one of the two CpG (TCGAGATGTCACACAGAGGTACGA; SEQ ID NO: 24) the amount of unmethylated C was estimated to 25% and 36% of total after 0.2 and 1 μM of 5-aza-2′deoxycytidine, respectively. For the other CpG (TCGAGATGTCACACAGAGGTACGA; SEQ ID NO: 24) the amount of unmethylated C was estimated to 35% and 45% after 0.2 and 1 μM of 5-aza-2′deoxycytidine, respectively.

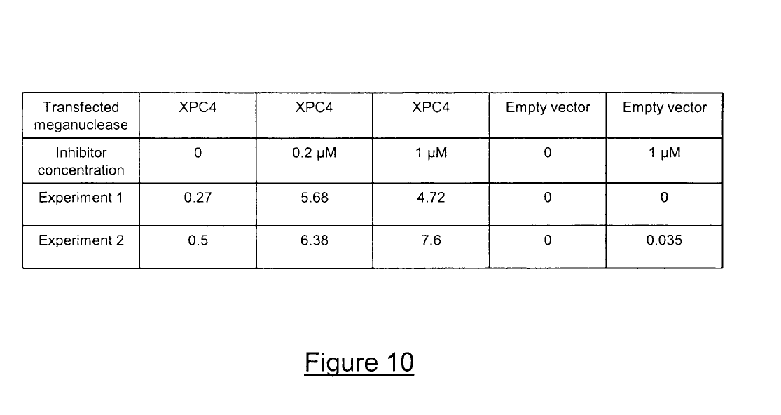

The rate of mutations induced by the XPC4 meganuclease in its cognate target was measured by deep sequencing. The region of the locus was amplified by PCR to obtain a specific fragment flanked by specific adaptator needed for HTS sequencing on the 454 sequencing system (454 Life Sciences). Results are presented in FIG. 10. 0.2-0.5% of PCR fragments carried a mutation in samples corresponding to cells transfected with the XPC4 meganuclease in the absence of 5-aza-2′deoxycytidine. In contrast, up to 7.6% of mutations were observed in samples treated with 5-aza-2′deoxycytidine. Mutagenesis was low or absent in cells transfected with empty vector and treated with 1 μM of 5-aza-2′deoxycytidine (FIG. 10).

Thus, it was observed that in the presence of 5-aza-2′deoxycytidine, there is a very strong increase in the rate of mutagenesis induced by meganuclease. Furthermore, this increase correlates with an actual demethylation of the XPC4 target. Therefore, it was concluded that demethylation and stimulation of mutagenesis are associated events, which both result from the presence of 5-aza-2′deoxycytidine.

c) Impact of siDNMT1 on Mutagenesis Induced by Meganuclease

To investigate the effect of DNA methylation on meganuclease cleavage, an engineered meganuclease called XPC4 (SEQ ID NO: 2) designed to cleave a DNA sequence 5′-TCGAGATGTCACACAGAGGTACGA-3′ (SEQ ID NO: 24) present in the Xeroderma Pigmentosum group C gene (XPC) was used. The XPC4 target contains two CpG motives, potentially methylated in cells. The impact of siRNA targeting the DNA methyltransferase DNMT1 gene on the methylation profile of these two CpG motives was measured, as well as on the cleavage efficiency of XPC4 target by the XPC4 meganuclease.

Materials and Methods

Cells Transfection

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS). The next day, cells were transfected with 5 μg of empty vector pCLS003 (SEQ ID NO: 65) and 1 nM or 5 nM of si_DNMT1 composed of mixture of two siRNA DNMT1_{—}1 (ACGGTGCTCATGCTTACAACC, SEQ ID NO: 66) and DNMT1_{—}2 (CCCAATGAGACTGACATCAAA, SEQ ID NO: 67) or with si_AS, a siRNA control with no known human target, using Lipofectamine 2000 as transfection reagent (Invitrogen) according to the manufacturer's protocol. The day after, cells were re-platted at the density of 1.2×10⁶ cells per 10 cm. 24 hours after, cells were transfected again with 1 nM or 5 nM of siDNMT1 or si_AS in presence of 3 μg of meganuclease expressing vector (pCLS2510; SEQ ID NO: 68) and 2 μg of empty vector or 5 μg of empty vector (SEQ ID NO: 65), with Lipofectamine 2000 transfection reagent (Invitrogen) according to the manufacturer's protocol.

Monitoring of DNA Methylation by Bisulfite Treatment and DNA Sequencing

To assess the level DNA methylation, DNA sequencing was performed after a bisulfite treatment according to the instructions of the manufacturer (EZ DNA methylation-Gold Kit, Zymo Research). After genomic DNA extraction, the XPC4 target locus was amplified by PCR with specific primers

F1:
(SEQ ID NO: 25)
5′-GTTGGTATAGATTAGTGGTTAGAGGTGTTTTG-3′
and
R1:
(SEQ ID NO: 26)
5′-CTTAAAACCCCTAACAACCAAAACCTTACC-3′.

The PCR product was sequenced directly with primers:

F2:
(SEQ ID NO: 27)
5′-GTGGGTATGTGTAGATTGTGTGTAYGGTGTG-3′
and
R2:
(SEQ ID NO: 28)
5′-CTCCAAATCTTCTTTCTTCTCCCTATCC-3′.

Monitoring of Meganuclease-Induced Mutagenesis by Deep Sequencing

After genomic DNA extraction, the XPC4 target locus was amplified with specific primers flanked by specific adaptator needed for HTS sequencing on the 454 sequencing system (454 Life Sciences)

F3:
(SEQ ID NO: 29)
5′-CCATCTCATCCCTGCGTGTCTCCGACTCAGTGCCAAGAGGCAAGAA
AATGTGCAGC-3′
and
R3:
(SEQ ID NO: 30)
5′-BiotineTEG/CCTATCCCCTGTGTGCCTTGGCAGTCTCAGGCTGG
GCATATATAAGGTGCTCAA-3′.

5,000 to 10,000 sequences per sample were analyzed.

Results

293H cells were transfected with XPC4 meganuclease or empty vector in presence of siRNA targeting DNMT1 gene or a siRNA control, at the concentration of 1 nM or 5 nM.

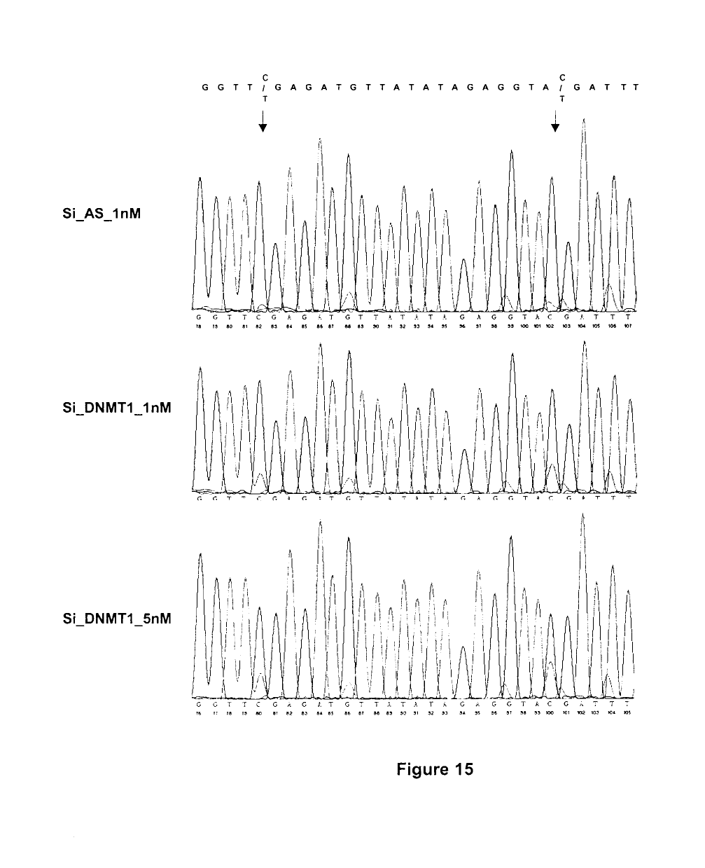

In order to determine the methylation status of the two CpG motives present in the XPC4 target sequence, genomic DNA was extracted, and treated with bisulfite. Bisulfite treatment is based on a chemical reaction of sodium bisulfite with DNA that converts unmethylated cytosines into uracil whereas methylated cytosines remain unchanged. DNA was then amplified by PCR and sequenced. Examples of sequences are shown in FIG. 15. In presence of si_AS, no cytosine conversion was observed in XPC4 target sequence, showing that both CpG were methylated in the vast majority of the cells. After transfection with si_DNMT1, we observed dual peaks in the chromatogram (FIG. 15), showing that in the treated cell population, the two CpG could be methylated or unmethylated. For one of the two CpG (TCGAGATGTCACACAGAGGTACGA; SEQ ID NO: 24) the amount of unmethylated C was estimated to 20 and 30% of total after 1 nM and 5 nM of siDNMT1, respectively. For the other CpG (TCGAGATGTCACACAGAGGTACGA; SEQ ID NO: 24) the amount of unmethylated C was estimated to 25 and 50% after 1 nM and 5 nM of si_DNMT1, respectively.

The rate of mutations induced by the XPC4 meganuclease in its cognate target was measured by deep sequencing. The region of the locus was amplified by PCR to obtain a specific fragment flanked by specific adaptator needed for HTS sequencing on the 454 sequencing system (454 Life Sciences). Results are presented in Table IIbis. 0.2-0.3% of PCR fragments carried a mutation in samples corresponding to cells transfected with the XPC4 meganuclease in the presence of non relevant siRNA (si_AS). In contrast, up to 7% of mutations were observed in samples treated with si_DNMT1. Mutagenesis was low or absent in cells transfected with empty vector and treated with 1 or 5 nM of si_DNMT1 (Table IIbis).

Thus, it was observed that in the presence of siRNA targeting the DNA methyltransferase, there is a very strong increase in the rate of mutagenesis induced by meganuclease. Furthermore, this increase correlates with an actual demethylation of the XPC4 target. Therefore, it was concluded that demethylation of the XPC4 target in vivo strongly enhance its cleavage by the XPC4 meganuclease.

TABLE IIbis
Impact of siRNAs targeting DNMT1 gene on mutagenesis of XPC4
meganuclease.

| | | Si_AS | Si_DNMT1 | Si_DNMT1 |
|---|---|---|---|---|
| Plasmid | Si_AS 1 nM | 5 nM | 1 nM | 5 nM |
| XPC4 | 0.3% | 0.2% | 4.8% | 7.6% |
| meganuclease | | | | |
| Empty vector | 0.005% | 0.011% | 0.022% | 0.055% |

d) Effect of 5-Aza-Deoxycytidine on Gene Targeting Induced by Meganuclease XPC4 In Vivo at its Endogenous Locus

Cell culture as well as general transfection conditions were described in “material and methods” section of part b) above. For this assay, 293H cells were co-transfected with 3 μg of XPC4 meganuclease expressing vector or empty vector and 2 μg of DNA repair matrix. The DNA repair matrix consists of a left and right arms corresponding to isogenic sequences of 1 kb located on both sides of the meganuclease recognition site. These two homology arms are separated by a heterologous fragment of 29 bp (sequence: AATTGCGGCCGCGGTCCGGCGCGCCTTAA, SEQ ID NO: 64). Two days post-transfection, cells were replated in 10 cm dish. Two weeks later, individual clones were picked and subsequently amplified in 96 wells plates for 3 days. 480 individual cellular clones were then analyzed per condition. DNA extraction was performed with the ZR-96 genomic DNA kit (Zymo research) according to the supplier's protocol. The detection of targeted DNA matrix integrations was performed by specific PCR amplification using the primers: XPC4_F4: 5′-TTAAGGCGCGCCGGACCGCGGC-3′ (SEQ ID NO: 41) (located within the 29 bp of heterologous sequence, i.e. SEQ ID NO: 64) and XPC4_R4: 5′-GATCATATCGTTGGGTTACGTCCCTG-3′ (located on the genomic sequence outside of the homology) (SEQ ID NO: 42).

Results

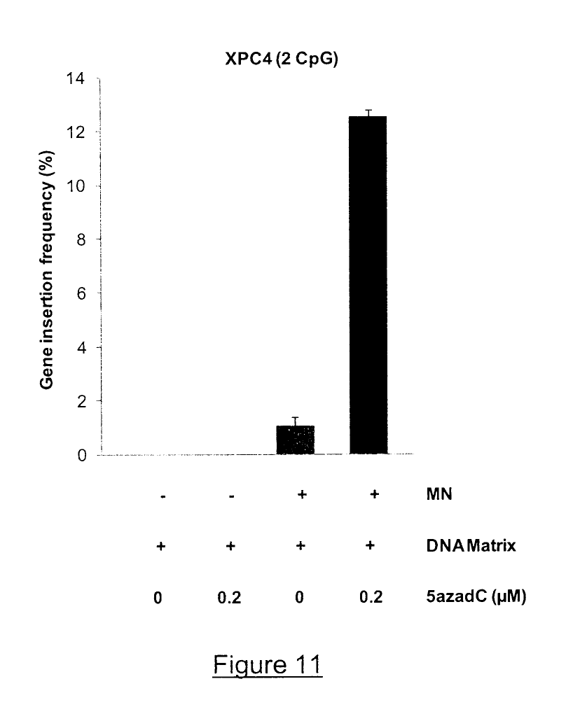

The rate of gene insertion events induced by the XPC4 meganuclease at its cognate target was quantified by measuring the ratio of PCR product carrying insertion/deletion events using a PCR-sequencing strategy as described in material and methods. As shown in FIG. 11, cells population treated with 5-aza-2′deoxycytidine (0.2 μM) exhibits higher rate of gene insertion events when co-transfected with the meganuclease expression vector and the repair matrix vector. Indeed, the analysis of individual cellular clones for targeted event revealed that in absence of 5-aza-2′deoxycytidine, targeted events could be detected in 1.05%±0.34 (n=2) of the transfected cells, while this frequency increases 12 fold reaching 12.5%±0.26 (n=2) when the cell population was treated with the same DNA methylase inhibitor. In contrast, no targeted events could be detected in absence of meganuclease with or without 5-aza-2′deoxycytidine treatment.

Thus, it was observed that treatment of the cell population with a DNA methylation inhibitor decreases the overall percentage of methylated CpG within the XPC4 meganuclease target. Moreover, the efficiency of the meganuclease is significantly increased in presence of 5-aza-2′deoxycytidine as shown by the increase of cell number in which targeted events occurred.

### Example 5

### Influence of DNA Methylation In Vitro and In Vivo on ADCY9 Meganuclease

a) Influence of DNA Methylation on the Binding Affinity and Nuclease Activity In Vitro of Engineered Meganuclease ADCY9 Towards its Specific DNA Target ADCY9.1

To further investigate the effect of DNA methylation on the binding affinity and nuclease activity of a meganuclease for its DNA target, an engineered meganuclease named ADCY9 specifically designed to cleave adenylate cyclase 9 gene was used. For that purpose, in vitro binding and cleavage assays were performed using recombinant ADCY9 (SEQ ID NO: 3) and its natural target ADCY9.1 containing either 0 methylated CG (composed of SEQ ID NO: 18+SEQ ID NO: 19) or 2 methylated CGs at positions −3a, −2b, respectively (composed of SEQ ID NO: 20+SEQ ID NO: 21).

Materials and Methods

Cloning, Overexpression and Purification of ADCY9

ADCY9 (SEQ ID NO: 3) was cloned, overexpressed and purified, according to the procedures previously described in Example 3.

Binding Assay

To determine the affinity of ADCY9 for its unmethylated and methylated specific target ADCY9.1, binding assays were performed according to the procedure described in example 3 using 5′ end fluorescein labeled unmethylated (composed of SEQ ID NO: 18+SEQ ID NO: 19) and methylated ADCY9.1 oligonucleotides (composed of SEQ ID NO: 20+SEQ ID NO: 21).

In Vitro Cleavage Assay

To investigate the influence of ADCY9.1 methylation on the nuclease activity of ADCY9, in vitro single turn over cleavage assays were performed with either unmethylated (composed of SEQ ID NO: 18+SEQ ID NO: 19) or methylated ADCY9.1 (composed of SEQ ID NO: 20+SEQ ID NO: 21) according to the procedure described in example 3.

Results

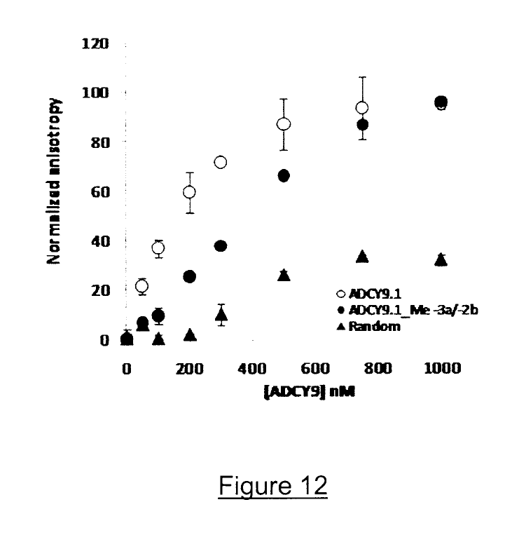

To investigate the influence of DNA methylation on the binding affinity of ADCY9 for its DNA target (ADCY9.1), the dissociation constant values (IQ) for methylated and unmethylated ADCY9.1 with ADCY9 were determined in vitro. Fluorescence anisotropy of fluorescein-labeled ADCY9.1 duplex was recorded in the presence of increasing amounts ADCY9 (FIG. 12). In the case of unmethylated ADCY9.1, fluorescence anisotropy increased and then leveled up at saturating concentration of ADCY9. This pattern was consistent with a binding equilibrium between ADCY9 and ADCY9.1. The dissociation constant of this binding equilibrium could be estimated to 190±19 nM.

Similarly, with fully methylated ADCY9.1, fluorescence anisotropy increased and then leveled up at saturating concentration of ADCY9. However, fluorescence anisotropy variation pattern was sigmoidal. The apparent K_{d} could be estimated to 431±30 nM.

These results indicated that the affinity of ADCY9 for methylated ADCY9.1 was significantly lower than for unmethylated ADCY9.1. Therefore, methylation of ADCY9.1 decreased its affinity for ADCY9.

In Vitro Cleavage Assay

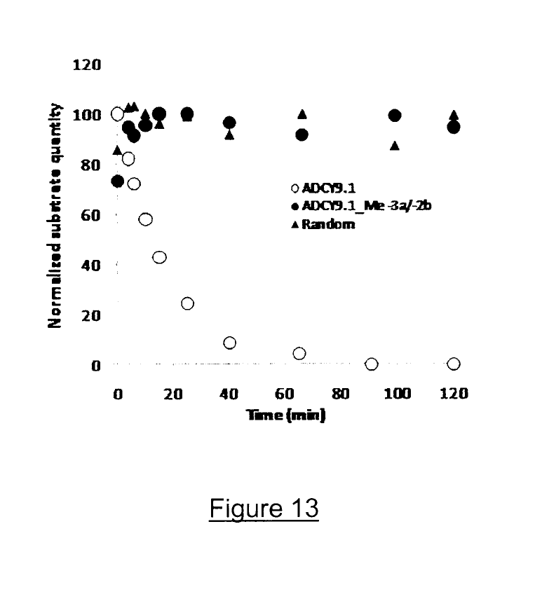

To test the influence of ADCY9.1 methylation on ADCY9 cleavage activity, single turn over cleavage assays were performed in vitro with unmethylated (composed of SEQ ID NO: 18+SEQ ID NO: 19) or with fully methylated forms of ADCY9.1 (composed of SEQ ID NO: 20+SEQ ID NO: 21) as substrates as described in example 3. In the case of unmethylated ADCY9.1, our results showed that the disappearance of ADCY9.1 substrate followed a monoexponential behavior that was characteristic of a first order process. The rate constant of this process was determined to be k=0.057±0.001 min⁻¹ (FIG. 13, open circles, table III). This indicated that ADCY9 efficiently cleaved ADCY9.1 with a k_{cat}=0.057±0.001 min⁻¹. In stark contrast, when fully methylated ADCY9.1 was assayed, no substrate disappearance was observed (FIG. 13, filled circles) even after 5 hours of reaction length (data not shown). This result indicated that ADCY9.1 methylation totally inhibited the nuclease activity of ADCY9.

Taken together, these results showed that methylation of ADCY9.1 at positions −3a, −2b strongly affected the cleavage activity of ADCY9. These results were consistent with the conclusions drawn in example 3 where it was found that methylation of cytosine-2b inhibited cleavage activity of I-Cre I.

TABLE III
K_{d} and k_{cat} of ADCY9 meganuclease for ADCY9.1 DNA targets.

| | ADCY9 | | |
|---|---|---|---|
| Meganuclease | | ADCY9.1_Me | |
| DNA target | ADCY9.1 | −3a/−2b | Random |
| K_{d} (nM) | 190 ± 19 | 431 ± 30 | >1000 |
| k_{cat} (min−1) | 0.057 ± 0.001 | <0.0001 | <0.0001 |

b) Effect of 5-Aza-Deoxycytidine on the Cleavage Efficiency Induced by Meganuclease ADCY9 In Vivo and on Gene Targeting at its Endogenous Locus

In this example, the impact of the DNA methylation in vivo was investigated on the meganuclease activity at endogenous locus. The ADCY9 target is in a CpG rich locus, with 61 CpG in 1 kb of surrounding sequence, and contains one CpG motif. This CpG motif is potentially methylated in cells. The engineered meganuclease called ADCY9 was used for these experiments. This meganuclease was designed to cleave the DNA sequence 5′-CCCAGATGTCGTACAGCAGCTTGG-3′ (SEQ ID NO: 18) present in the human adenylate cyclase 9 gene mRNA (NM_{—}001116.2). The DNA target contains 1 CpG motif that appears to be methylated in human 293H cell line. The impact of a methylase inhibitor was evaluated (i) on the methylation profile of this CpG motif, and (ii) on the efficiency of the meganuclease to promote DSB-induced mutagenesis.

Materials and Methods

Cell Transfection

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS). The cells were pre-treated with 5-aza-deoxycytidine at 0.2 μM or 1 μM, 48 hours before transfection and the treatment was maintained 48 hours post-transfection. The medium was changed every day. The cells were transfected with 5 μg of DNA plasmids encoding meganuclease using Lipofectamine 2000 transfection reagent (Invitrogen) according to the manufacturer's protocol.

Monitoring the DNA Methylation Status in 293H Human Cells.

The procedure as described in Example 4 was followed to assess the level of DNA methylation, except that we used specific primers to amplify the sequence surrounded the ADCY9 meganuclease recognition site. Primers ADCY9_F1 GTAGGTTTAGGAYGGTAGTTATTYGTAGGAG (SEQ ID NO: 43) and ADCY9_R1 CCCTTAACATTCACRATCCCTCTATAATC (SEQ ID NO: 44) were used. PCR products were sequenced directly with primer ADCY9_F2 GAGTTYGTTAAGGAGATGATGYGYGTGGTGG (SEQ ID NO: 45).

Meganuclease-Induced Mutagenesis Assay

The efficiency of the meganuclease to promote mutagenesis at its endogenous recognition site was evaluated by sequencing the DNA surrounding the meganuclease cleavage site.

Two days post-transfection, genomic DNA was extracted. 200 ng of genomic DNA were used to amplify (PCR amplification) the endogenous locus surrounding the meganuclease cleavage site. PCR amplification is performed to obtain a fragment flanked by specific adaptor sequences [adaptor A: 5′-CCATCTCATCCCTGCGTGTCTCCGAC-NNNN-3′ (SEQ ID NO: 46) and adaptor B, 5′-CCTATCCCCTGTGTGCCTTGGCAGTCTCAG-3′ (SEQ ID NO: 47)] provided by the company offering sequencing service (GATC Biotech AG, Germany) on the 454 sequencing system (454 Life Sciences). The primers sequences used for PCR amplification were: ADCY9_F3: 5′-CCATCTCATCCCTGCGTGTCTCCGACTCAG-NNNN-ACAGCAGCATCGAGAAGATC-3′ (SEQ ID NO: 48) and ADCY9_R3: 5′-CCTATCCCCTGTGTGCCTTGGCAGTCTCAG-ATGCTGCCATCCACCTGGACG-3′ (SEQ ID NO: 49). Sequences specific to the locus are underlined. The sequence NNNN in primer F1 is a Barcode sequence (Tag) needed to link the sequence with a PCR product. The percentage of PCR fragments carrying insertion or deletion at the meganuclease cleavage site is related to the mutagenesis induced by the meganuclease through NHEJ pathway in a cell population, and therefore correlates with the meganuclease activity at its endogenous recognition site. 5000 to 10000 sequences were analyzed per conditions.

Meganuclease-Induced Gene Targeting Assay

Cell culture as well as general transfection conditions were described in Example 4. For this assay, 293H cells were co-transfected with 5 μg of ADCY9 meganuclease expressing vector and 2 μg of DNA repair matrix. The DNA repair matrix consists of a left and right arms corresponding to isogenic sequences of 1 kb located on both sides of the meganuclease recognition site. These two homology arms are separated by a heterologous fragment of 29 bp (sequence: AATTGCGGCCGCGGTCCGGCGCGCCTTAA, SEQ ID NO: 64). Two days post-transfection, cells were replated in 10 cm dish. Two weeks later, individual clones were picked and subsequently amplified in 96 wells plate for 3 days. 480 individual cellular clones were then analyzed per condition. DNA extraction was performed with the ZR-96 genomic DNA kit (Zymo research) according to the supplier's protocol. The detection of targeted DNA matrix integrations was performed by specific PCR amplification using the primers ADCY9_F4: 5′-TTAAGGCGCGCCGGACCGCGGC-3′ (specific to the 29 bp of heterologous sequence) (SEQ ID NO: 50) and ADCY9_R4: 5′-TACGAGTTTAAGACCAGCCTTGGC-3′ (specific to a genomic sequence located outside of the homology arm) (SEQ ID NO: 51).

Results

The ADCY9 target recognizes by the engineered meganuclease contains one CG dinucleotides sequence (CpG) which could potentially contains a methylated cytosine (5′-CCCAGATGTCGTACAGCAGCTTGG-3′, SEQ ID NO: 18). Analysis of the methylation status by bisulfite technique shows that 100% of this CpG were methylated in the 293H cell population that was studied, while treatment of the cell population with 0.2 and 1 μM of 5-aza-2′-deoxycytidine reduced to 40% the amount of methylated CpG within the same DNA target.

Moreover, the rate of mutagenesis induced by the ADCY9 meganuclease at its cognate target was quantified by measuring the ratio of PCR product carrying insertion/deletion events using a PCR-sequencing strategy as described in material and methods. As shown in table IV, when the cell population was transfected with the ADCY9 meganuclease expression vector, 0.16%±0.06 (n=2) of the PCR fragments carried a mutation in absence of 5-aza-2′-deoxycytidine treatment. In contrast, up to 0.48%±0.01 (n=2) of mutations was observed in samples treated with 5-aza-2′deoxycytidine. Mutagenesis was extremely low (0.03%) in cells transfected with empty vector and treated with 1 μM of 5-aza-2′deoxycytidine.

TABLE IV
Impact of 5-aza-2′-deoxycytidine on mutagenesis induced by ADCY9
meganuclease at its recognition site (*, from 2 independent
experiments).

| Meganuclease | 0 μM | 0.2 μM | 1 μM |
|---|---|---|---|
| ADCY9 | 0.16% ± 0.06* | 0.48% ± 0.01* | 0.36 |
| Empty vector | 0.02 | 0.03 | 0.03 |

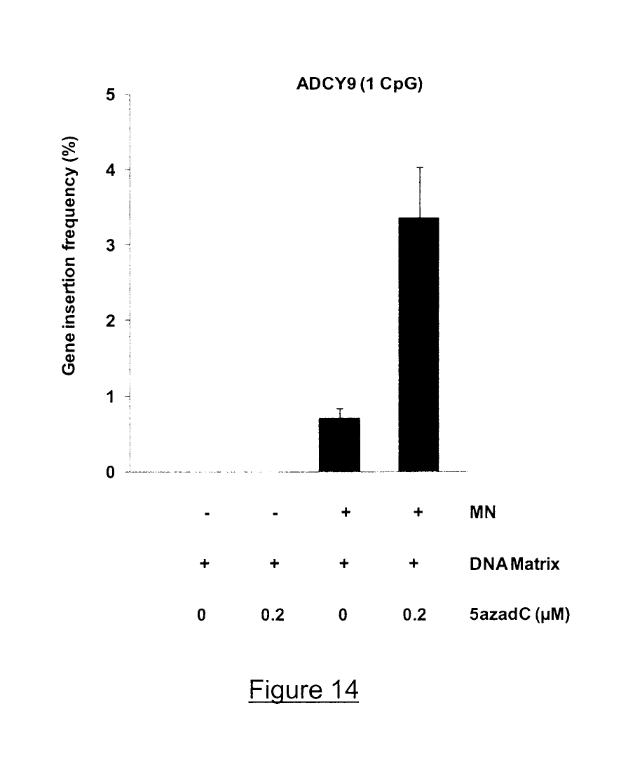

Similarly, as shown in FIG. 14, cells population treated with 5-aza-2′deoxycytidine (0.2 μM) exhibits higher rate of gene insertion events when co-transfected with the meganuclease expression vector and the repair matrix vector. Indeed, the analysis of individual cellular clone for targeted event revealed that in absence of 5-aza-2′deoxycytidine, targeted events could be detected in 0.70%±0.14 (n=2) of the transfected cells, while this frequency increases 4.8 fold reaching 3.37%±0.66 (n=2) when the cell population was treated with the same DNA methylase inhibitor. In contrast, no targeted events could be detected in absence of meganuclease with or without 5-aza-2′deoxycytidine treatment.

Thus, treatment of the cell population with a DNA methylation inhibitor decreases the overall percentage of methylated CpG within the ADCY9 meganuclease target. Moreover, the efficiency of the meganuclease is significantly increased in presence of 5-aza-2′deoxycytidine as shown by the increase of either the rate of induced mutagenesis, either the frequency of cells in which targeted events occurred. Together with the in vitro data showing that methylation inhibits binding and cleavage of the ADCY9 target by the ADCY9 meganuclease, these data show that methylation of the ADCY9 target in vivo impaired the meganuclease activity at its endogenous recognition site, resulting in a low efficacy. However, the treatment of the cells with drugs that abolish or decrease DNA methylation strongly enhances its efficacy.

### Example 6

### Methylase Inhibitor 5-Aza-2′-Deoxycytidine does not Affect Meganuclease-Induced Gene Targeting in Absence of Methylated CpG Dinucleotides within its DNA Target

In this example, the impact of the DNA methylation in vivo on the meganuclease activity at endogenous locus was investigated. The engineered meganucleases called RAG (Single chain, SEQ ID NO: 61) and CAPNS1 (heterodimer, SEQ ID NO: 62+SEQ ID NO: 63) were used for these experiments. These meganucleases were designed to cleave the DNA sequence 5′-TTGTTCTCAGGTACCTCAGCCAGC-3′ (SEQ ID NO: 52) presents in the human RAG1 gene (NM_{—}000448.2) and the 5′ UTR of the human CAPNS1 (Calpain small subunit 1) gene (NM_{—}001749.2) 5′-CAGGGCCGCGGTGCAGTGTCCGAC-3′ (SEQ ID NO: 53), respectively. The RAG target does not contain CpG dinucleotide sequence. The CAPNS1 target contains 3 CpGs, but is embedded in a CpG island. Since this CpG island is in the 5′ UTR of an highly expressed gene, one can hypothesize that it is actually unmethylated. The impact of a methylase inhibitor was evaluated on the efficiency of the meganuclease to promote DSB-induced mutagenesis.

Materials and Methods

Cell Transfection

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS). The cells were pre-treated with 5-aza-deoxycytidine at 0.2 μM or 1 μM, 48 hours before transfection and the treatment was maintained 48 hours post-transfection. The medium was changed every day. The cells were transfected with 3 μg of DNA plasmids encoding meganuclease for RAG or 2.5 μg of each monomer CAPNS1 using Lipofectamine 2000 transfection reagent (Invitrogen) according to the manufacturer's protocol.

Monitoring the DNA Methylation Status in 293H Human Cells.

The procedure described in example 5 was followed to assess the level of DNA methylation, except that specific primers were used to amplify the sequence surrounded the CAPNS1 meganuclease recognition site. Primers CAPNS1_F1 GGGTGTTTTTATTTAGATTTGAGGGGTG (SEQ ID NO: 54) and CAPNS1_R1 CTAAAAATCRATTCCACTACCRCTCCC (SEQ ID NO: 55) were used. PCR products were sequenced directly with primer CAPNS1_F2 GTTAGGGYGGGATTAAGATTTTYGG (SEQ ID NO: 56).

Meganuclease-Induced Mutagenesis Assay

The efficiency of the meganuclease to promote mutagenesis at its endogenous recognition site was evaluated by sequencing the DNA surrounding the meganuclease cleavage site. Two days post-transfection, genomic DNA was extracted. 200 ng of genomic DNA were used to amplify (PCR amplification) the endogenous locus surrounding the meganuclease cleavage site. PCR amplification is performed to obtain a fragment flanked by specific adaptor sequences [adaptor A: 5′-CCATCTCATCCCTGCGTGTCTCCGAC-NNNN-3′(SEQ ID NO: 46) and adaptor B, 5′-CCTATCCCCTGTGTGCCTTGGCAGTCTCAG-3′ (SEQ ID NO: 47)] provided by the company offering sequencing service (GATC Biotech AG, Germany) on the 454 sequencing system (454 Life Sciences). The primers sequences used for PCR amplification were:

### RAG_F1: 5′-CCATCTCATCCCTGCGTGTCTCCGACTCAG-NNNN-GGCAAAGATGAATCAAAGATTCTGTCCT (SEQ ID NO: 57) and

RAG_R1: 5′-CCTATCCCCTGTGTGCCTTGGCAGTCTCAG-GATCTCACCCGGAACAGCTTAAATTTC-3′ (SEQ ID NO: 58) and CAPNS1_J3: 5′-CCATCTCATCCCTGCGTGTCTCCGACTCAG-NNNN-CGAGTCAGGGCGGGATTAAG (SEQ ID NO: 59) and CAPNS1_R3: 5′-CCTATCCCCTGTGTGCCTTGGCAGTCTCAG-CGAGACTTCACGGTTTCGCC-3′ (SEQ ID NO: 60). Sequences specific to the locus are underlined. The sequence NNNN in primer F1 is a Barcode sequence (Tag) needed to link the sequence with a PCR product. The percentage of PCR fragments carrying insertion or deletion at the meganuclease cleavage site is related to the mutagenesis induced by the meganuclease through NHEJ pathway in a cell population, and therefore correlates with the meganuclease activity at its endogenous recognition site. 5000 to 10000 sequences were analyzed per conditions.

Results

The CAPNS1 target recognizes by the engineered meganuclease contains three CG dinucleotides sequences (CpG) which could potentially contain a methylated cytosine (5′-CAGGGCCGCGGTGCAGTGTCCGAC-3′, (SEQ ID NO: 53). Analysis of the methylation status by bisulfite technique shows that none of these CpGs were methylated in the 293H cell population that was studied. The rate of mutagenesis induced by the CAPNS1 and RAG meganuclease at its cognate target was quantified by measuring the ratio of PCR product carrying insertion/deletion events using a PCR-sequencing strategy as described in material and methods. As shown in table V, when the cell population was transfected with the RAG meganuclease expression vector, 0.75%±0.02 (n=2) of the PCR fragments carried a mutation in absence of 5-aza-2′-deoxycytidine treatment. No increase of mutations was observed in samples treated with 0.2 μM of 5-aza-2′deoxycytidine 0.71%±0.17 (n=2) and treated with 1 μM of 5-aza-2′deoxycytidine 0.59%±0.31 (n=2). Mutagenesis was extremely low (0.04%) in cells transfected with empty vector and treated with 1 μM of 5-aza-2′deoxycytidine.

When the cell population was transfected with the CAPNS1 meganuclease expression vector, 6.1%±0.43 (n=2) of the PCR fragments carried a mutation in absence of 5-aza-2′-deoxycytidine treatment. No increase of mutations was observed in samples treated with 0.2 μM of 5-aza-2′deoxycytidine, 6.55%±0.77 (n=2) or treated with 1 μM of 5-aza-2′deoxycytidine 4.34%±0.95 (n=2). Mutagenesis was low (0.22%±0.25) in cells transfected with empty vector and treated with 1 μM of 5-aza-2′deoxycytidine.

Thus, treatment of the cell population with a DNA methylation inhibitor does not affect in vivo meganuclease-induced gene targeting in absence of methylated CpG dinucleotides within its DNA target.

TABLE V
Impact of 5-aza-2′-deoxycytidine on mutagenesis induced by RAG
and CAPNS1 meganucleases at their recognition site (*, from
2 independent experiments).

| Meganuclease | 0 μM | 0.2 μM | 1 μM |
|---|---|---|---|
| RAG | 0.76% ± 0.02* | 0.71% ± 0.17* | 0.59% ± 0.3* |
| Empty vector | ND | ND | 0.04 ± 0.05 |
| CAPNS1 | 6.10% ± 0.43* | 6.55% ± 0.77* | 4.34% ± 0.95* |
| Empty vector | ND | ND | 0.22% ± 0.25* |

### REFERENCES

Hinnen, A., et al. Proc Natl Acad Sci USA. 1978 75: 1929-33.
Rothstein R J Methods Enzymol. 1983; 101:202-11.
Thomas, K. R. and Capecchi, M. R. Cell, 1987 51, 503-512.
Capecchi et al. Nat Med 2001 7(10): 1086-90.
Paques, F. and J. E. Haber Microbiol Mol Biol Rev 1999 63(2): 349-404.
Sung and Klein, Nat Rev Mol Cell Biol. 2006 October; 7(10):739-50. Epub 2006 Aug. 23
Roeder G S Genes Dev 1997, 11, 2600-2621
van Gent et al. Nat Rev Genet 2001 2(3): 196-206.
Capecchi, M. R., Science, 1989, 244, 1288-1292
Smithies, O., Nature Medicine, 2001, 7, 1083-1086
Rouet, P., et al. (1994). Proc Natl Acad Sci USA 91(13): 6064-8.
Rouet, P., et al. (1994). Mol Cell Biol 14(12): 8096-106.
Choulika, A., et al. (1995). Mol Cell Biol 15(4): 1968-73.
Dujon et al, Basic Life Sci. 1986; 40:5-27.
Haber, Bioessays. 1995 July; 17(7):609-20.
Posfai et al. Nucleic Acids Res 1999 27(22): 4409-15.
Sargent et al. Mol Cell Biol 1997 17: 267-277
Cohen-Tannoudji et al. Mol Cell Biol 1998 18: 1444-1448
Donoho et al. Mol Cell Biol 1998 18: 4070-4078
Gouble et al J Gene Med 2006 8(5): 616-22.
Puchta, H., et al. Proc Natl Acad Sci USA 1996 93(10): 5055-60.
Siebert, R. and H. Puchta Plant Cell 2002 14(5): 1121-31.
Paques et al. Curr Gen Ther. 2007 7:49-66
Arnould et al. J Mol Biol. 2007 371:49-65
Smith et al. NAR 2006 34:e149
Grizot et al. NAR 2009 37:5405
Bird et al. J Cell Sci 1995 Suppl 19, 37-9
Noyer-Weidner et al. EXS 1993 64, 39-108.
Cooper et al. J Biol Chem 1993 268, 11823-9.
Binz et al. Mycologia 1998 90 (5): 785-790
Selker et al. Nature 2003 422 (6934): 893-897
Tucker K L et al. 2001 Neuron 30 (3): 649-52
Monk et al, Development 1987 99:371-382.
Kafri et al, Genes & Dev. 1992 6:705-714.
Bird et al, EMBO J. 1987 6:999-1004.
Carroll et al. New York: W.H. Freeman and CO. 2008 p. 403. ISBN 0-7167-6887-9.
Mann et al. Genome Biol. 2002 3 (2): 1003.1-.4.
Weaver et al. Epigenetics 2007 2 (1): 22-8.
Cooper et al. Hum Genet 1988 78, 151-5
Rideout et al. Science 1990 249, 1288-90
Fearon et al. FASEB J 1992 6, 2783-90
Greenblatt et al Cancer Res 1994 54, 4855-78
Lapeyre et al. Biochem Biophys Res Commun 1979 87, 698-705
Gama-Sosa et al. Nucleic Acids Res 1983, 11, 6883-94
Feinberg et al. Cancer Res 1988 48, 1159-61
Bird et al. Trends Genet 1995 11, 94-100
Kisseljova et al. Int J Oncol 1998 12, 203-9
Sasaki et al. EXS 1993 64, 469-86
Collick, et al. Development, 1988 104, 235-44
Studier et al. Proc Natl Acad Sci USA. 1988 July; 85(13):4677-81
Bickle T A (1982) Cold Spring Harbor Monogr. Ser. 14, 85-108
Shapiro et al., 1973 J Biol Chem. 1973 Jun. 10; 248(11):4060-4
Frommer et al., 1992 Proc Natl Acad Sci USA. 1992 Mar. 1; 89(5):1827-31.
Cheng et al., Cancer cell, Cancer Cell 2004 6(2): 151-8.
Momparler, R. L. Semin Hematol 2005 42(3 Suppl 2): S9-16.
Zhou et al., J Mol Biol 2002 321(4): 591-9.
Porteus et al. Nat Biotechnol. 2005 23:967-973
Arimondo et al. Mol Cell Biol. 2006 26:324-333
Simon et al. NAR 2008 36:3531-3538
Eisenschmidt et al. NAR 2005 33:7039-7047
Kalish and Glazer Ann NY Acad Sci 2005 1058: 151-61
Liu et al. NAR 2009 37:6378-6388
Majumdar et al. J. Biol. Chem 2008 283, 17:11244-11252
Stoddard, Quarterly Reviews of Biophysics, 2006, 38:49-95
Chevalier, B. S. and B. L. Stoddard, Nucleic Acids Res., 2001, 29, 3757-3774
Chevalier, et al., Nat. Struct. Biol., 2001, 8, 312-316
Chevalier et al., J. Mol. Biol., 2003, 329, 253-269
Spiegel et al., Structure, 2006, 14, 869-880
Moure et al., J. Mol. Biol., 2003, 334, 685-69
Silva et al., J. Mol. Biol., 1999, 286, 1123-1136
Bolduc et al., Genes Dev., 2003, 17, 2875-2888
Ichiyanagi et al., J. Mol. Biol., 2000, 300, 889-901
Moure et al., Nat. Struct. Biol., 2002, 9, 764-770
Chevalier et al., Mol. Cell., 2002, 10, 895-905
Epinat et al., Nucleic Acids Res, 2003, 31, 2952-62
Sussman et al., J. Mol. Biol., 2004, 342, 31-41
Seligman et al., Genetics, 1997, 147, 1653-1664
Arnould et al., J. Mol. Biol., 2006, 355, 443-458
Rosen et al., Nucleic Acids Res., 2006, 34, 4791-4800
Doyon et al., J. Am. Chem. Soc., 2006, 128, 2477-2484
Gimble et al., J. Mol. Biol., 2003, 334, 993-1008
Ashworth et al., Nature, 2006, 441, 656-659
Chames et al., Nucleic Acids Res., 2005, 33, e178
Pingoud, A. and G. H. Silva Nat Biotechnol 2007 25(7): 743-4.
Boch, J., H. Scholze, et al. (2009). Science 326(5959): 1509-12.
Christian, M., T. Cermak, et al. (2010). Genetics 186(2): 757-61.
Li, T., S. Huang, et al. (2010). Nucleic Acids Res 39(1): 359-72.
Moscou, M. J. and A. J. Bogdanove (2009). Science 326(5959): 1501.
Wang J, Kim H H, Yuan X, Herrin D L (1997). Nucleic Acids Res 1997, 25:3767-3776

1-4. (canceled)

5. A method for improving cleavage of DNA from a chromosomal locus in a cell by an engineered rare-cutting endonuclease sensitive to methylation, the method comprising:
(i) identifying at the chromosomal locus a DNA target sequence of more than 14 base pairs in length wherein the DNA target sequence comprises no more than 3 CpG motifs;
(ii) engineering the rare-cutting endonuclease; and
(iii) contacting the DNA target sequence with the rare-cutting endonuclease, to obtain cleavage of the DNA target sequence.

6. The method of claim 5, wherein the rare-cutting endonuclease sensitive to methylation is a meganuclease.

7. The method of claim 5, wherein the rare-cutting endonuclease sensitive to methylation is a meganuclease from the LAGLIDADG family.

8. The method of claim 5, wherein the rare-cutting endonuclease sensitive to methylation is a meganuclease derived from an I-CreI meganuclease.

9. The method of claim 5, wherein the DNA target sequence comprises no CpG motif in position −2 to +2.

10. The method of claim 5 wherein the DNA target sequence comprises no CpG motif in position +5 to +3 or in position −2 to +2.

11. The method of claim 5, wherein the DNA target sequence comprises no CpG motif in positions ±10 to ±8, ±5 to ±3 or −2 to +2.

12. The method of claim 5, wherein the DNA target sequence comprises no more than two CpG dinucleotides.

13. The method of claim 5, wherein the DNA target sequence comprises no more than one CpG dinucleotide.

14. The method of claim 5, wherein the DNA target sequence comprises no CpG dinucleotide.

15. The method of claim 5, wherein the cell is a eukaryotic cell.

16. The method of claim 5, wherein the cell is a mammalian cell.

17. A method for improving cleavage of DNA from a chromosomal locus in a chosen cell type or organism, by an engineered rare-cutting endonuclease sensitive to methylation, the method comprising:
(i) determining a CpG content of a potential DNA target sequence;
(ii) determining a methylation level of the DNA target sequence in at least one cell type related to the chosen cell type or organism;
(iii) selecting at least one potential DNA target sequence displaying no methylation;
(iv) engineering the rare-cutting endonuclease; and
(v) contacting the DNA target sequence with the rare-cutting endonuclease, to obtain cleavage of the DNA target sequence.

18. The method of claim 17, wherein the rare-cutting endonuclease sensitive to methylation is a meganuclease.

19. The method of claim 17, wherein the rare-cutting endonuclease sensitive to methylation is a meganuclease from the LAGLIDADG family.

20. The method of claim 17, wherein the rare-cutting endonuclease sensitive to methylation is a meganuclease derived from an I-CreI meganuclease.

21. The method of claim 17, wherein the potential DNA target sequence displaying no methylation is a CpG island.

22. The method of claim 17, wherein the methylation level is assayed in the chosen cell type.

23. The method of claim 17, wherein the cell is a eukaryotic cell.

24. The method of claim 17, wherein the cell is a mammalian cell.

25. A method to select a target cell type for a rare-cutting endonuclease, the rare-cutting endonuclease cleaving a DNA target sequence comprising at least one CpG dinucleotide, the method comprising:
(i) determining a methylation level of the DNA target sequence in several cell types;
(ii) selecting a cell type displaying no methylation; and
(iii) contacting the DNA target sequence with the rare-cutting endonuclease.

26. The method of claim 25, wherein the rare-cutting endonuclease is a meganuclease.

27. The method of claim 25, wherein the rare-cutting endonuclease is a meganuclease from the LAGLIDADG family.

28. The method of claim 25, wherein the rare-cutting endonuclease is a meganuclease derived from an I-CreI meganuclease.

29. The method of claim 25, wherein the DNA target sequence is a CpG island.

30. The method of claim 25, wherein the cell is a eukaryotic cell.

31. The method of claim 25, wherein the cell is a mammalian cell.

32-38. (canceled)

39. An isolated polynucleotide that is more efficiently cleaved by a rare-cutting endonuclease.

40. A vector or genetic construct comprising the polynucleotide of claim 39.

41. A cell comprising the polynucleotide of claim 39 or comprising a vector or genetic construct comprising the polynucleotide of claim 39.

42. A kit comprising the isolated polynucleotide of claim 39 and at least one rare-cutting endonuclease and optionally instructions for using the rare-cutting endonuclease, buffer(s), salt(s), cofactor(s), positive or negative control polynucleotide(s), and/or target polynucleotide(s).

43. The method of claim 5, wherein the CpG motifs are methylated and the DNA target sequence is treated with an agent inhibiting methylation.

44. The method of claim 5, wherein the rare-cutting endonuclease sensitive to methylation is a TALEN.

45. The method of claim 17, wherein the rare-cutting endonuclease sensitive to methylation is a TALEN.

46. The method of claim 25, wherein the rare-cutting endonuclease is a TALEN.


SEQUENCE LISTING (73 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - protein, 354 aa, Artificial Sequence
  Description of Artificial Sequence Synthetic polypeptide
MANTKYNEEFLLYLAGFVDGDGSIIAQIKPNQSHKFKHALQLTFKVTQKTQRRWFLDKLVDEIGVGYVQDSGSVSNYILSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSHKFKHQLSLAFQVTQKTQRRWFLDKLVDRIGVGYVRDRGSVSDYILSKIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 3 - protein, 354 aa, Artificial Sequence
  Description of Artificial Sequence Synthetic polypeptide
MANTKYNEEFLLYLAGFVDGDGSIIAQIKPNQSYKFKHQLSLTFRVTQKTQRRWFLDKLVDEIGVGYVRDSGSVSNYDLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPDQSYKFKHQLGLTFQVTQKTQRRWFLDKLVDRIGVGYVRDRGSVSDYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 4 - DNA, 32 bp, Artificial Sequence
  Description of Artificial Sequence Synthetic oligonucleotide
ggggtcaaaacgtcgtacgacgttttgagggg

SEQ ID NO: 5 - DNA, 32 bp, Artificial Sequence
  Description of Artificial Sequence Synthetic oligonucleotide
cccctcaaaacgtcgtacgacgttttgacccc

SEQ ID NO: 6 - DNA, 32 bp, Artificial Sequence; identical to SEQ ID NO: 4
  Description of Artificial Sequence Synthetic oligonucleotide

SEQ ID NO: 7 - DNA, 32 bp, Artificial Sequence; identical to SEQ ID NO: 5
  Description of Artificial Sequence Synthetic oligonucleotide

SEQ ID NO: 8 - DNA, 32 bp, Artificial Sequence; identical to SEQ ID NO: 4
  Description of Artificial Sequence Synthetic oligonucleotide

SEQ ID NO: 9 - DNA, 32 bp, Artificial Sequence; identical to SEQ ID NO: 5
  Description of Artificial Sequence Synthetic oligonucleotide

SEQ ID NO: 10 - DNA, 32 bp, Artificial Sequence; identical to SEQ ID NO: 4
  Description of Artificial Sequence Synthetic oligonucleotide

SEQ ID NO: 11 - DNA, 32 bp, Artificial Sequence; identical to SEQ ID NO: 5
  Description of Artificial Sequence Synthetic oligonucleotide

SEQ ID NO: 12 - DNA, 32 bp, Artificial Sequence; identical to SEQ ID NO: 4
  Description of Artificial Sequence Synthetic oligonucleotide

SEQ ID NO: 13 - DNA, 32 bp, Artificial Sequence; identical to SEQ ID NO: 5
  Description of Artificial Sequence Synthetic oligonucleotide

SEQ ID NO: 14 - DNA, 24 bp, Artificial Sequence
  Description of Artificial Sequence Synthetic oligonucleotide
tcgagatgtcacacagaggtacga

SEQ ID NO: 15 - DNA, 24 bp, Artificial Sequence
  Description of Artificial Sequence Synthetic oligonucleotide
tcgtacctctgtgtgacatctcga

SEQ ID NO: 16 - DNA, 24 bp, Artificial Sequence
  Description of Artificial Sequence Synthetic oligonucleotide
tcgagatgtcacacagaggtacga

SEQ ID NO: 17 - DNA, 24 bp, Artificial Sequence
  Description of Artificial Sequence Synthetic oligonucleotide
tcgtacctctgtgtgacatctcga

SEQ ID NO: 18 - DNA, 24 bp, Artificial Sequence
  Description of Artificial Sequence Synthetic oligonucleotide
cccagatgtcgtacagcagcttgg

SEQ ID NO: 19 - DNA, 24 bp, Artificial Sequence
  Description of Artificial Sequence Synthetic oligonucleotide
ccaagctgctgtacgacatctggg

SEQ ID NO: 20 - DNA, 24 bp, Artificial Sequence
  Description of Artificial Sequence Synthetic oligonucleotide
cccagatgtcgtacagcagcttgg

SEQ ID NO: 21 - DNA, 24 bp, Artificial Sequence
  Description of Artificial Sequence Synthetic oligonucleotide
ccaagctgctgtacgacatctggg

SEQ ID NO: 22 - protein, 167 aa, Artificial Sequence
  Description of Artificial Sequence Synthetic polypeptide
MANTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSPAAD

SEQ ID NO: 23 - DNA, 24 bp, Artificial Sequence
  Description of Artificial Sequence Synthetic oligonucleotide
tcaaaacgtcgtacgacgttttga

SEQ ID NO: 24 - DNA, 24 bp, Artificial Sequence
  Description of Artificial Sequence Synthetic oligonucleotide
tcgagatgtcacacagaggtacga

SEQ ID NO: 25 - DNA, 32 bp, Artificial Sequence
  Description of Artificial Sequence Synthetic oligonucleotide
gttggtatagattagtggttagaggtgttttg

SEQ ID NO: 26 - DNA, 30 bp, Artificial Sequence
  Description of Artificial Sequence Synthetic oligonucleotide
cttaaaacccctaacaaccaaaaccttacc

SEQ ID NO: 27 - DNA, 31 bp, Artificial Sequence
  Description of Artificial Sequence Synthetic oligonucleotide
gtgggtatgtgtagattgtgtgtayggtgtg

SEQ ID NO: 28 - DNA, 28 bp, Artificial Sequence
  Description of Artificial Sequence Synthetic oligonucleotide
ctccaaatcttctttcttctccctatcc

SEQ ID NO: 29 - DNA, 56 bp, Artificial Sequence
  Description of Artificial Sequence Synthetic oligonucleotide
ccatctcatccctgcgtgtctccgactcagtgccaagaggcaagaaaatgtgcagc

SEQ ID NO: 30 - DNA, 54 bp, Artificial Sequence
  Description of Artificial Sequence Synthetic oligonucleotide
cctatcccctgtgtgccttggcagtctcaggctgggcatatataaggtgctcaa

SEQ ID NO: 31 - DNA, 24 bp, Artificial Sequence
  Description of Artificial Sequence Synthetic oligonucleotide
tcaaaacgtcgtgagacagtttgg

SEQ ID NO: 32 - DNA, 24 bp, Artificial Sequence
  Description of Artificial Sequence Synthetic oligonucleotide
ccaaactgtctcacgacgttttga

SEQ ID NO: 33 - DNA, 24 bp, Artificial Sequence
  Description of Artificial Sequence Synthetic oligonucleotide
tcaaaacgtcgtgagacagtttgg

SEQ ID NO: 34 - DNA, 24 bp, Artificial Sequence
  Description of Artificial Sequence Synthetic oligonucleotide
tcaaaacgtcgtgagacagtttgg

SEQ ID NO: 35 - DNA, 24 bp, Artificial Sequence
  Description of Artificial Sequence Synthetic oligonucleotide
tcaaaacgtcgtgagacagtttgg

SEQ ID NO: 36 - DNA, 24 bp, Artificial Sequence
  Description of Artificial Sequence Synthetic oligonucleotide
ccaaactgtctcacgacgttttga

SEQ ID NO: 37 - DNA, 24 bp, Artificial Sequence
  Description of Artificial Sequence Synthetic oligonucleotide
ccaaactgtctcacgacgttttga

SEQ ID NO: 38 - DNA, 24 bp, Artificial Sequence
  Description of Artificial Sequence Synthetic oligonucleotide
ccaaactgtctcacgacgttttga

SEQ ID NO: 39 - DNA, 24 bp, Artificial Sequence
  Description of Artificial Sequence Synthetic oligonucleotide
gagagagagagagagagagagaga

SEQ ID NO: 40 - DNA, 24 bp, Artificial Sequence
  Description of Artificial Sequence Synthetic oligonucleotide
tctctctctctctctctctctctc

SEQ ID NO: 41 - DNA, 22 bp, Artificial Sequence
  Description of Artificial Sequence Synthetic oligonucleotide
ttaaggcgcgccggaccgcggc

SEQ ID NO: 42 - DNA, 26 bp, Artificial Sequence
  Description of Artificial Sequence Synthetic oligonucleotide
gatcatatcgttgggttacgtccctg

SEQ ID NO: 43 - DNA, 31 bp, Artificial Sequence
  Description of Artificial Sequence Synthetic oligonucleotide
gtaggtttaggayggtagttattygtaggag

SEQ ID NO: 44 - DNA, 29 bp, Artificial Sequence
  Description of Artificial Sequence Synthetic oligonucleotide
cccttaacattcacratccctctataatc

SEQ ID NO: 45 - DNA, 31 bp, Artificial Sequence
  Description of Artificial Sequence Synthetic oligonucleotide
gagttygttaaggagatgatgygygtggtgg

SEQ ID NO: 46 - DNA, 30 bp, Artificial Sequence
  Description of Artificial Sequence Synthetic oligonucleotide
ccatctcatccctgcgtgtctccgacnnnn

SEQ ID NO: 47 - DNA, 30 bp, Artificial Sequence
  Description of Artificial Sequence Synthetic oligonucleotide
cctatcccctgtgtgccttggcagtctcag

SEQ ID NO: 48 - DNA, 54 bp, Artificial Sequence
  Description of Artificial Sequence Synthetic oligonucleotide
ccatctcatccctgcgtgtctccgactcagnnnnacagcagcatcgagaagatc

SEQ ID NO: 49 - DNA, 51 bp, Artificial Sequence
  Description of Artificial Sequence Synthetic oligonucleotide
cctatcccctgtgtgccttggcagtctcagatgctgccatccacctggacg

SEQ ID NO: 50 - DNA, 22 bp, Artificial Sequence
  Description of Artificial Sequence Synthetic oligonucleotide
ttaaggcgcgccggaccgcggc

SEQ ID NO: 51 - DNA, 24 bp, Artificial Sequence
  Description of Artificial Sequence Synthetic oligonucleotide
tacgagtttaagaccagccttggc

SEQ ID NO: 52 - DNA, 24 bp, Artificial Sequence
  Description of Artificial Sequence Synthetic oligonucleotide
ttgttctcaggtacctcagccagc

SEQ ID NO: 53 - DNA, 24 bp, Artificial Sequence
  Description of Artificial Sequence Synthetic oligonucleotide
cagggccgcggtgcagtgtccgac

SEQ ID NO: 54 - DNA, 28 bp, Artificial Sequence
  Description of Artificial Sequence Synthetic oligonucleotide
gggtgtttttatttagatttgaggggtg

SEQ ID NO: 55 - DNA, 27 bp, Artificial Sequence
  Description of Artificial Sequence Synthetic oligonucleotide
ctaaaaatcrattccactaccrctccc

SEQ ID NO: 56 - DNA, 25 bp, Artificial Sequence
  Description of Artificial Sequence Synthetic oligonucleotide
gttagggygggattaagattttygg

SEQ ID NO: 57 - DNA, 62 bp, Artificial Sequence
  Description of Artificial Sequence Synthetic oligonucleotide
ccatctcatccctgcgtgtctccgactcagnnnnggcaaagatgaatcaaagattctgtcct

SEQ ID NO: 58 - DNA, 57 bp, Artificial Sequence
  Description of Artificial Sequence Synthetic oligonucleotide
cctatcccctgtgtgccttggcagtctcaggatctcacccggaacagcttaaatttc

SEQ ID NO: 59 - DNA, 54 bp, Artificial Sequence
  Description of Artificial Sequence Synthetic oligonucleotide
ccatctcatccctgcgtgtctccgactcagnnnncgagtcagggcgggattaag

SEQ ID NO: 60 - DNA, 50 bp, Artificial Sequence
  Description of Artificial Sequence Synthetic oligonucleotide
cctatcccctgtgtgccttggcagtctcagcgagacttcacggtttcgcc

SEQ ID NO: 61 - protein, 354 aa, Artificial Sequence
  Description of Artificial Sequence Synthetic polypeptide
MANTKYNEEFLLYLAGFVDGDGSIIAQINPNQSSKFKHRLRLTFYVTQKTQRRWFLDKLVDEIGVGYVRDSGSVSQYVLSEIKPLHNFLTQLQPFLELKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKKFLLYLAGFVDSDGSIIAQIKPRQSNKFKHQLSLTFAVTQKTQRRWFLDKLVDRIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 62 - protein, 167 aa, Artificial Sequence
  Description of Artificial Sequence Synthetic polypeptide
MANTKYNKEFLLYLAGFVDSDGSIIAQIKPRQSYKFKHQLRLTFYVTQKTQRRWFLDKLVDEIGVGYVEDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 63 - protein, 167 aa, Artificial Sequence
  Description of Artificial Sequence Synthetic polypeptide
MANTKYNKEFLLYLAGFVDGDGSIVAQIKPNQRAKFKHQLSLTFQVTQKTQRRWLLDKLVDEIGVGYVQDSGSVSNYRLSEIKPLHNFLTQLQPFLKLKQKQANLVLKIIEQLPSAKESPDKFLEVCTWADQIAALNDSKTRKTTSETVRAVLDSLSEKKKPSPAAD

SEQ ID NO: 64 - DNA, 29 bp, Artificial Sequence
  Description of Artificial Sequence Synthetic oligonucleotide
aattgcggccgcggtccggcgcgccttaa

SEQ ID NO: 65 - DNA, 5428 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 66 - DNA, 21 bp, Artificial Sequence
  Description of Artificial Sequence Synthetic oligonucleotide
acggtgctcatgcttacaacc

SEQ ID NO: 67 - DNA, 21 bp, Artificial Sequence
  Description of Artificial Sequence Synthetic oligonucleotide
cccaatgagactgacatcaaa

SEQ ID NO: 68 - DNA, 6220 bp, Artificial Sequence
  Description of Artificial Sequence Synthetic polynucleotide
tcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgccgaattcatggccaataccaaatataacgaagagttcctgctgtacctggccggctttgtggacggtgacggtagcatcatcgctcagattaaaccaaatcagtctcataagtttaaacatgctctacagttgacctttaaggtgactcaaaagacccagcgccgttggtttctggacaaactagtggatgaaattggcgttggttacgtacaggatagtggatccgtttccaactacatcttaagcgaaatcaagccgctgcacaacttcctgactcaactgcagccgtttctggaactgaaacagaaacaggcaaacctggccctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccaaatcaatctcacaagttcaaacaccagctctccttggcctttcaagtcactcagaagacacaaagaaggtggttcttggacaaattggttgataggattggtgtgggctatgtcagagacagaggctctgtgtcagactacatcctgtctaaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagagatc

SEQ ID NO: 69 - DNA, 40 bp, Artificial Sequence
  Description of Artificial Sequence Synthetic oligonucleotide
tgggttcgagatgttatatagaggtacgatttagtttgga

SEQ ID NO: 70 - DNA, 40 bp, Artificial Sequence
  Description of Artificial Sequence Synthetic oligonucleotide
tgggttygagatgttatatagaggtaygatttagtttgga

SEQ ID NO: 71 - DNA, 30 bp, Artificial Sequence
  Description of Artificial Sequence Synthetic oligonucleotide
ggttygagatgttatatagaggtaygattt

SEQ ID NO: 72 - DNA, 30 bp, Artificial Sequence
  Description of Artificial Sequence Synthetic oligonucleotide
ggttcgagatgttatatagaggtacgattt

SEQ ID NO: 73 - protein, 6 aa, Artificial Sequence
  Description of Artificial Sequence Synthetic 6xHis tag
HHHHHH